(19) United States
(12) Patent Application Publication
(10) Pub. No.: US 2025/0387473 A1
(43) Pub. Date: Dec. 25, 2025

(54) MAKING PROGRAMMED CELL-DERIVED VESICLES

(71) Applicant: University of Kentucky Research Foundation, Lexington, KY (US)

(72) Inventors: Chris Richards, Versailes, KY (US); Jill Kolesar, Washington, DC (US)

(21) Appl. No.: 19/244,820

(22) Filed: Jun. 20, 2025

Related U.S. Application Data
(60) Provisional application No. 63/662,135, filed on Jun. 20, 2024.

(51) Int. Cl. A61K 39/39; A61K 9/50; A61K 39/00; A61P 37/04; C12N 5/0786

(52) U.S. Cl. CPC A61K 39/39; A61K 9/5068; A61P 37/04; C12N 5/0645; A61K 2039/55555; A61K 2039/575; A61K 2039/585; C12N 2510/00

(57) ABSTRACT

The presently-disclosed subject matter includes programmed cell-derived vesicles (CDVs), methods of making programmed CDVs, and methods of using programmed CDVs.

### RELATED APPLICATIONS

This application claims priority from U.S. Provisional Application Ser. No. 63/662,135 filed Jun. 20, 2024, the entire disclosure of which is incorporated herein by this reference.

### TECHNICAL FIELD

The presently-disclosed subject matter generally relates to cell-derived vesicles and methods of making such cell-derived vesicles that are programmed for targeting specific cell types and for beneficially modulating target cells. In particular, certain embodiments of the presently-disclosed subject matter relate to cell-derived vesicles engineered from cellular membranes to have specific immunomodulatory properties, including the capability to bidirectionally modulate immune cell polarization. These programmed cell-derived vesicles are decorated with specific membrane-bound ligands that can be targeted toward specific cell types, including immune cells. In some embodiments, these programmed cell-derived vesicles are engineered to enhance immune cell reprogramming toward a proinflammatory phenotype. In this regard, the vesicles can be macrophage-engineered vesicles useful for re-polarizing M2 macrophages toward a proinflammatory phenotype.

### INTRODUCTION

Vesicle-based nanoparticles including exosomes, microvesicles, and liposomes have been leveraged as potential therapeutic tools for cancer treatment due to their ability to specifically target the tumor environment and their ability to elicit a tumor-specific immune response.

Exosomes are nano-sized (diameter 40-150 nm) extracellular vesicles (EVs) released by the cells through normal physiological processes and contain a wide range of biological cargo including proteins and RNA which can be used to communicate information to target cells. Exosome targeting specificity can be harnessed to controllably deliver therapeutics both in cell culture and in vivo. In addition, exosomes released by antigen-presenting cells (APCs) including dendritic cells and macrophages can also activate the immune system. Despite these promising characteristics, low production yields and difficulties in separating exosomes from biological solutions still pose barriers to their use in clinical applications.

Liposomes, synthetically generated lipid bilayer vesicles, have been used as an alternative to exosomes in therapeutic delivery. While liposomes can be produced in large quantities, they lack the inherent biocompatibility seen with endogenous exosomes and are prone to immune clearance when delivered in vivo.

Recently, cell-derived vesicles (CDVs) obtained by fragmenting cellular membranes have been found to mimic many of the positive attributes of exosomes and have shown promise as therapeutic delivery platforms because they can be produced in high yield, exhibit targeting specificity and have low immunogenicity when delivered in vivo.

Vesicles derived from antigen-presenting cells offer additional potential avenues for therapeutics because of their ability to serve as immunomodulatory platforms. Macrophages are the most abundant immune effector cells present in the tumor microenvironment and exhibit a continuum of functional states between pro-inflammatory (M1) and anti-inflammatory (M2) polarization. M1 macrophages are known to have anti-tumoral properties including engulfing and destroying phagocytosed tumor cells and activating different components of the immune system. However, M2 macrophages stimulate tumor angiogenesis and inhibit the anti-tumor immune response mediated by T-cells.

Along with small molecule immunomodulators and extracellular vesicles, cell-derived vesicles from M1 macrophages have been shown to alter the polarization of tumor-associated macrophages which play a role in chemotherapy resistance and promote metastasis. While these therapeutic approaches show promise, they suffer from unique challenges. The efficacy of small molecule-based therapeutics is limited by their rapid degradation and inability to preferentially target tumor-associated macrophages (TAMs) in vivo. While EVs are biostable, exhibit targeting specificity, and can modulate macrophage phenotype in the tumor microenvironment, EV-based therapies are challenged by their low production yield. Cell-derived vesicle-based therapies overcome several challenges that limit other nanoscale therapeutics, but CDVs would be more effective with more specific targeting and higher efficacy in repolarizing anti-inflammatory macrophages to a proinflammatory phenotype.

An approach has been tried for enhancing specific targeting of another type of particle, a synthetic therapeutic-loaded lipid-polymer-based nanoparticle. In particular, the nanoparticle was engineered with 1,2-distearoyl-sn-glycero-3-phosphoethanolamine-poly(ethylene glycol) (DSPE-PEG)-mannose and monophosphoryl lipid A (MPLA) to simultaneously improve their dendritic cell targeting and ability to execute enhanced immune responses. Similarly, toll-like receptor 7 and 8 (TLR7/8) agonists presenting nanoparticles have been generated using poly(ethylene glycol)-poly(lactic acid) (PEG-PLA) to enhance the immunomodulatory properties of nanoparticles.

The fragmentation of cellular membranes to form cell derived vesicles has been accomplished using a variety of techniques including nitrogen cavitation, cellular extrusion, and sonication. In all cases, the vesicles formed through these processes are a mix of membranes from various organelles.

Overall, the existing art has a number of limitations. Naturally secreted exosomes suffer from low production yields, making them impractical for scalable therapeutic use. Additionally, existing approaches often lack targeting specificity and immunomodulatory potency, leading to off-target effects and systemic immune activation. Furthermore, synthetic nanoparticles and small molecule therapies are prone to rapid degradation, immune clearance, and toxicity.

Accordingly, there remains a need in the art for improved cell-derived vesicles (CDVs) and methods of making and using CDVs, which allow for improved production yield, targeting specificity, and therapeutic efficacy.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes programmed cell-derived vesicles (CDVs), methods of making programmed CDVs, and methods of using programmed CDVs. A CDV is a vesicle generated from the membrane of a cell. For example, a vesicle can be generated from a cell that is a tumor cell, a dendritic cell, a macrophage, or another type of cell.

In some embodiments of the presently-disclosed subject matter, a method of making a programmed CDV is provided, which method comprises (a) obtaining a donor cell from which the CDV will be generated; (b) wherein the donor cell is isolated from an organelle of interest and/or wherein the donor cell overexpresses a ligand of interest on the surface of the cell; and (c) fragmenting the membrane of the donor cell and allowing the fragmented membrane to assemble into a CDV, wherein the CDV is an organelle-specific CDV and/or wherein the CDV displays the ligand of interest.

In some embodiments, the method of making a programmed CDV comprises (a) obtaining a donor cell from which the CDV will be generated; (b) overexpressing a ligand of interest on the surface of the donor cell; and (c) fragmenting the membrane of the donor cell and allowing the fragmented membrane to assemble into a CDV displaying the ligand of interest on its surface. In some embodiments, the method further comprises overexpressing the ligand of interest by transfecting the donor cell with a plasmid for expressing the ligand of interest. In some embodiments of the method, the ligand of interest is selected from the group consisting of CD54, TNF-α, CpG-ODN, ICOS, and combinations thereof. In some embodiments of the method, the ligand of interest is a polarization-inducing ligand and/or a targeting-enhancing ligand. In some embodiments of the method the ligand of interest targets a specific cell type and optionally modulates the specific target cell type. In some embodiments of the method the specific target cell type is an immune cell or a tumor cell. In some embodiments of the method the ligand of interest has specific immunomodulatory properties, including the capability to bidirectionally modulate immune cell polarization. In some embodiments of the method the ligand of interest enhances cell reprogramming toward a proinflammatory phenotype. In some embodiments of the method the CDV is an MEV useful for re-polarizing M2 macrophages toward a proinflammatory phenotype. In some embodiments of the method the ligand of interest promotes cellular uptake of the CDV displaying the ligand of interest.

In some embodiments, the method of making a programmed CDV comprises isolating a donor cell from an organelle of interest, from which the CDV will be generated; and fragmenting the membrane of the donor cell and allowing the fragmented membrane to assemble into an organelle-specific CDV. In some embodiments of the method, the organelle of interest is selected from the group consisting of endoplasmic reticulum (ER), plasma membrane (PM), mitochondria. In some embodiments, the resulting CDV is an ER-derived MEV (erMEV), a PM-derived MEV, or a mitochondria-derived MEV.

In some embodiments of the method of making a programmed CDV, the donor cell is a tumor cell, a dendritic cell, or a macrophage; however, other types of cells can be used. In some embodiments, when the donor cell is a macrophage, it can be a bone marrow-derived macrophage (M0). In some embodiments, the macrophage (M0) is programmed to overexpress a ligand of interest on its surface.

In some embodiments of the method of making a programmed CDV, the donor cell is a ligand-of-interest-expressing macrophage (M0) and the method further comprises polarizing the ligand-of-interest-expressing macrophages (M0) to a M1 macrophage that is pro-inflammatory. In some embodiments of the method, polarizing the donor cell to a M1 phenotype comprises contacting the donor cell with lipopolysaccharide (LPS) and interferon gamma (IFN-γ). In some embodiments, the method of making a programmed CDV further comprises incubating a donor macrophage with an MEV of a desired phenotype to achieve reprogramming of the donor macrophage to the desired phenotype.

In some embodiments of the method of making a programmed CDV, when a ligand of interest is overexpressed in the donor cell, the ligand of interest is selected for interaction with a target of interest. In some embodiments, the ligand of interest selectively binds the target of interest. In some embodiments, the target of interest is on a target cell. In some embodiments, the target of interest is in an in vivo environment. In some embodiments, the ligand of interest is selected from the group consisting of CD54, TNF-α, CpG-ODN, ICOS, and combinations thereof.

In some embodiments of the method of making a programmed CDV, when the donor cell is isolated from an organelle of interest, there is a surface feature of the donor cell that interacts with a target of interest. In some embodiments, the surface feature selectively binds the target of interest. In some embodiments, the target of interest is on a target cell. In some embodiments, the target of interest is in an in vivo environment. In some embodiments, the donor cell is isolated from endoplasmic reticulum. In some embodiments, the CDV is an endoplasmic reticulum-derived macrophage-engineered vesicle (erMEV).

In some embodiments of the method of making a programmed CDV, cargo is encapsulated within the CDV. In this regard, in some embodiments, the method further comprises suspending the fragmented membrane in an assembly solution comprising cargo such that the CDV encapsulates the cargo during assembly. The cargo can be, for example, a therapeutic agent and/or a diagnostic agent. In some embodiments, the cargo is a dye, small molecule, nucleotide, polypeptide, a gene editing component, or combinations thereof.

The presently-disclosed subject matter further includes a method of shifting a target macrophage phenotype. In some embodiments of the method, the target macrophage is shifted to an M1 phenotype. Some embodiments of the method comprise (a) contacting the target macrophage with a cell derived vesicle (CDV) displaying a ligand of interest, selected from the group consisting of CD54, TNF-α, CpG-ODN, ICOS, and combinations thereof; (b) contacting the target macrophage with an of endoplasmic reticulum-derived macrophage-engineered vesicle (erMEV); (c) contacting the target macrophage with an MEV derived from an M1 macrophage; or (d) combinations thereof. In some embodiments, the target macrophage is in an in vivo environment. In some embodiments, the in vivo environment is the site of a cancer in a subject.

The presently-disclosed subject matter further includes a cell-derived vesicle (CDV) as disclosed herein. In some embodiments, the CDV is prepared by a method as disclosed herein.

In some embodiments, the CDV comprises a membrane from a donor cell overexpressing a ligand of interest, such that the CDV displays the ligand of interest on its surface. In some embodiments, the donor cell is transfected with a plasmid for expressing the ligand of interest. In some embodiments, the ligand of interest is selected from the group consisting of CD54, TNF-α, CpG-ODN, ICOS, and combinations thereof. In some embodiments, the ligand of interest is a polarization-inducing ligand and/or a targeting-enhancing ligand. In some embodiments, the ligand of interest targets a specific cell type and optionally modulates the specific target cell type. In some embodiments, the specific target cell type is an immune cell or a tumor cell. In some embodiments, the ligand of interest has specific immunomodulatory properties, including the capability to bidirectionally modulate immune cell polarization. In some embodiments, the ligand of interest enhances cell reprogramming toward a proinflammatory phenotype. In some embodiments, the CDV is an MEV useful for re-polarizing M2 macrophages toward a proinflammatory phenotype. In some embodiments, the ligand of interest promotes cellular uptake of the CDV displaying the ligand of interest. In some embodiments, the CDV is polarized to an M1 phenotype. In some embodiments, the CDV is polarized to an M2 phenotype. In some embodiments, the ligand of interest is selected for interaction with a target of interest. In some embodiments, the ligand of interest selectively binds the target of interest. In some embodiments, the target of interest is on a target cell. In some embodiments, the target of interest is in an in vivo environment. In some embodiments, the ligand of interest is selected from the group consisting of CD54, TNF-α, CpG-ODN, ICOS, and combinations thereof. In some embodiments, the CDV further comprises cargo encapsulated within the CDV. In some embodiments, the cargo is a therapeutic agent and/or a diagnostic agents. In some embodiments, the cargo is a dye, small molecule, nucleotide, polypeptide, a gene editing component, or combinations thereof.

In some embodiments, the CDV comprises a membrane from a donor cell isolated from an organelle of interest, which that the CDV is organelle-specific. In some embodiments, the organelle of interest is selected from the group consisting of endoplasmic reticulum (ER), plasma membrane (PM), mitochondria. In some embodiments, the CDV is an is an endoplasmic reticulum-derived macrophage-engineered vesicle (erMEV), a plasma membrane-derived macrophage-engineered vesicle (pmMEV), or a mitochondria-derived macrophage-engineered vesicle. In some embodiments, a surface feature of the donor cell interacts with a target of interest. In some embodiments, the surface feature selectively binds the target of interest. In some embodiments, the target of interest is on a target cell. In some embodiments, the target of interest is in an in vivo environment. In some embodiments, the donor cell is isolated from endoplasmic reticulum. In some embodiments, the CDV is an endoplasmic reticulum-derived macrophage-engineered vesicle (erMEV). In some embodiments, the CDV further comprises cargo encapsulated within the CDV. In some embodiments, the cargo is a therapeutic agent and/or a diagnostic agents. In some embodiments, the cargo is a dye, small molecule, nucleotide, polypeptide, a gene editing component, or combinations thereof.

### BRIEF DESCRIPTION OF THE DRAWINGS

The features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1. Schematic diagram illustrating the approach of generating programmed MEVs from programmed bone marrow-derived macrophages. Macrophages (M0) are first programmed to overexpress desired ligands on their surface and then polarized into either pro-inflammatory macrophages (M1). Programmed MEVs were generated using nitrogen cavitation and MEVs were purified from cellular fragments by serial centrifugation. M2 macrophages were then treated with programmed MEVs to shift their polarization toward the pro-inflammatory phenotype.

FIG. 2A-2D. Macrophage Engineered Vesicles (MEVs) Characterization FIG. 2A) Size distribution of MEVs obtained from Nanoparticle Tracking Analysis (NTA). Nitrogen cavitation of the macrophages results in the generation of MEVs of effective diameter between 50-200 nm. FIG. 2B) Sodium dodecyl sulfate-polyacrylamide gel electrophoresis (SDS-PAGE) analysis of the protein content of MEVs compared to M1 macrophages. FIG. 2C) Validation of exosome marker proteins in MEVs. Equal amounts of total proteins extracted from MEVs, M1 macrophages, and M2 macrophages were immunoblotted for CD106, CD54, CD63, CD9, CD81, MHCII, and CD11b. FIG. 2D) Wide-field fluorescence image of MEVs labeled with a lipophilic dye DiI. Scale bar=100 μm.

FIG. 3. Transmission electron microscopy micrograph of negatively stained Raw 264.7-derived vesicles. Vesicles were measured to have a diameter ranging from 25-150 nm, with an average diameter of 70±20 nm. Vesicle were observed to primarily possess a relatively spherical or oblong morphology. Deformation of the vesicles may be the result of sample preparation for TEM.

FIG. 4. Series of images showing DiI-labeled MEVs uptake by M2 BMDMs. Vesicles were added after the first time point in real time and images were taken for 2 h every 10 min. A 40× air objective was used with 561 nm excitation. Vesicles were labeled using the lipophilic dye, DiI.

FIG. 5A-5H. Reprogramming macrophage polarization by MEVs. FIG. 5A-5F: Immunostaining of iNOS (M1 macrophage marker) and CD206 (M2 macrophage marker) in M2 macrophages after incubation with 10⁸, 10⁹, 10¹⁰ and 10¹¹ MEVs for 24 h. Each data point is the average of at least 5 experiments (n =5). The data are presented as the mean±SEM. FIG. 5G-5H: Measurement of the proinflammatory cytokines including IFN-γ, IL-10, IL-12p60, IL-1β, IL-6, KC/GRO, and TNF-α released by M2 macrophages after incubating them with 1×10¹¹ MEVs in a time-dependent manner. Each data point is the average of at least 3 experiments (n=3). The data are presented as the mean±SEM.

FIG. 6A-6C. Cytokine content of MEVs (FIG. 6A) Quantification of pro-inflammatory cytokine present on MEVs. MEV were freeze-ruptured (MEV-FR) in liquid nitrogen to assess cytokines present inside of the MEVs. (FIG. 6B) A dose response study showing the pro-inflammatory cytokine-TNF-α released by M2 macrophages after incubation with different concentration of IFN-γ for 24 hours. (FIG. 6C) Quantification of pro-inflammatory cytokine-TNF-α released by M2 macrophages after incubation with 40 pg/mL IFN-γ which is representative of the cytokine present on 1×10¹¹ MEVs, 1×10¹¹ MEVs and M1 polarizing stimulants (LPS (20 ng/mL)+IFN-γ (20 ng/mL)) for 24 hours. Supernatants were assayed in triplicate using a mouse TNF-α V-PLEX cytokine assay kit from Meso Scale Discovery.

FIG. 7A-7E. Proteolytic digestion of membrane proteins present on MEVs eliminates the reprogramming capability of M2 macrophages toward an M1 phenotype. FIG. 7A: Western blotting to compare the presence of membrane-anchored proteins in MEVs treated with and without proteinase-K. FIG. 7B: Widefield fluorescence images of DiI labeled proteinase-K treated MEVs (pkt-MEVs) and untreated MEVs delivered to M2 macrophages after 1.5 h show the reduction in uptake of proteinase-K treated MEVs. FIG. 7C-7D: Comparison of delivery of fluorescent labeled MEVs (red) and fluorescent labeled pkt-MEVs (black) to M2 macrophages. FIG. 7E: TNF-α is released by M2 macrophages in a dose-dependent manner after 24 h of interaction with increasing concentrations of MEV or pkt-MEVs. Each data point is the average of at least 3 experiments (n=3). The data is presented as the mean±SEM. A Two-Sample t-Test w as used to determine the statistical significance between the endpoints. *p<0.01 indicates that the results are statistically significant.

FIG. 8. Experimental and computational workflow to identify MEV gene targets that promote M1 or disfavor M2. A list of localized proteins in the cell membrane and function as receptors was generated from the UniProt database. Pathways from the STRING database, KEGG, and our own curated database were generated to form a network of interconnected genes. Additionally, a list of proteins from an antibody experiment done on M1 was available. Cross-referencing these lists, from differentially expressed genes, pm-localized genes, network nodes, and antibody data, potential targets for M1 polarization can be narrowed down.

FIG. 9A-9D. TNF-α programmed nanovesicles cause M2 macrophages to repolarize more toward the M1 phenotype. FIG. 9A) Fluorescence image of HEK cells transfected with a mouse GFP-tagged TNF-α plasmid. Green fluorescence in the cell membrane indicated a clear overexpression of TNF-α on the cell membrane. FIG. 9B) IFN-γ, IL-10, IL-12p60, IL-1β, IL-6, KC/GRO, and TNF-α released by M2 macrophages after incubation with TNF-α programmed HEK-cell derived nanovesicles (P(TNF)-HNVs) and HNVs. FIG. 9C) A fluorescence image of macrophages transfected with a mouse GFP-tagged TNF-α plasmid. FIG. 9D) Comparison of IFN-γ, IL-10, IL-12p60, IL-1β, IL-6, KC/GRO, and TNF-α released by M2 macrophages incubated with P(TNF)-MEVs and regular M1 macrophage-derived vesicles (MEVs). Each data point is the average of at least 3 experiments (n=3). The data is presented as the mean±SEM. *p<0.01 indicates that the results are statistically significant.

FIG. 10. Western blot analysis of M1 and M2 macrophages compared to MEV treated M2 macrophages for p65 translocation.

FIG. 11A-11D. CCL5-programmed nanovesicles for M2 to M1 macrophage reprogramming. FIG. 11A: Chemokine expression image of the chemokine antibody array for MEVs and M2 macrophages. Dark spots in the images indicate the presence of the specific chemokine. FIG. 11B: Comparison of mean pixel integrated density measurements between chemokines present on MEVs and M2 macrophages. FIG. 11C: Western blotting shows that programmed HEK cells express more CCL5 than regular HEK cells. Accordingly, vesicles generated from programmed HEK cells express a greater CCL5 concentration. FIG. 11D: CCL5-programmed vesicles derived from HEK stimulate M2 BMDMs to produce more KC/GRO. Each data point is the average of at least 3 experiments (n=3). The data is presented as the mean±SEM. *p<0.01 indicates that the results are statistically significant.

FIG. 12. CCL5-programmed MEVs treated M2 BMDMs produce higher TNF-α compared M2 BMDMs incubated with regular MEVs.

FIG. 13A-13D. ICAM-1 programmed nanovesicles mediated M2 to M1 macrophage reprogramming. FIG. 13A: Western blotting analysis reveals that CD54 transfected programmed M1 macrophages (P(CD54)-M1) express more CD54 than non-transfected M1 macrophages. As a result, vesicles derived from programmed macrophages (P(CD54)-MEVs) have a higher concentration of ICAM-1. FIG. 13B: A bar graph displaying the relative fold of CD54 expression in M1 macrophages, programmed M1 macrophages, MEVs, and P(CD54)-MEVs. CD54 expressions are normalized to Na⁺K⁺ ATPase, a plasma membrane marker. FIG. 13C-13D: CD54-programmed vesicles exhibit higher repolarization efficiency compared to regular nanovesicles. Each data point is the average of at least 3 experiments (n=3). The data is presented as the mean±SEM. *p<0.01 indicates that the results are statistically significant.

FIG. 14A-14F. Dose response studies for the cytokine released by M2 macrophages that had been incubated with an increasing concentration of MEVs or CD54-programmed MEVs.

FIG. 15A-15D. Programming MEVs with TLR-ligands for enhanced M2 macrophage reprogramming toward an M1 phenotype. FIG. 15A: Size distribution graph for MEVs, CpG oligonucleotide incorporated MEVs (cpg-MEVs), and Pam3CSK4 decorated MEVs (pam-MEVs) from Nanoparticle Tracking Analysis. The mean diameter of MEVs (black), cpg-MEVs (red), and pam-MEVs (blue) is 127, 135, and 146 nm, respectively. FIG. 15B: Fluorescence image of rhodamine-labeled Pam3CSK4 decorated MEVs demonstrating successful MEV programming via Pam3CSK4 decoration. FIG. 15C: Comparison of the repolarization efficiency of Pam3CSK4 ligand decorated MEVs compared to regular MEVs. FIG. 15D: Comparison of the repolarization efficiency of cpg-ODN ligand incorporated MEVs compared to regular MEVs. Each data point is the average of at least 3 experiments (n=3). The data is presented as the mean±SEM.

FIG. 16. Schematic for generating endoplasmic reticulum and plasma membrane specific macrophage engineered vesicles (MEVs). (FIG. 16A) Isolation of bone marrow cells from femur and tibia of mice followed by incubation at 37° C. for 7 days to differentiate them into fully developed bone marrow derived macrophages. (FIG. 16B) Nitrogen cavitation of M1 Macrophages at 600 psi for 20 minutes. (FIG. 16C) Ultracentrifugation to isolate vesicles between the interfaces of OptiPrep solutions and the relative positions of different fractions isolated. MEVs were generated using mouse bone marrow derived proinflammatory (M1) macrophages. (FIG. 16D) Vesicle fraction isolation and purification.

FIG. 17A-17D. Macrophage engineered vesicles characterization. (FIG. 17A) Western blot of the different MEV fractions collected from the interfaces to confirm the presence of endoplasmic reticulum (er) and plasma membrane (pm) markers. Anti-sodium potassium ATPase antibody was used to bind with the sodium potassium ATPase, a plasma membrane marker (upper panel). An anti-calnexin antibody was used to bind with the er marker, calnexin, in the gradients (lower panel). The plasma membrane marker was found in the cell lysate and fractions 5 to 7 and the er marker were in the cell lysate and fractions 3 to 5. Therefore, fractions 3 and 4 contain only the er vesicles; fractions 6 and 7 contain only the plasma membrane vesicles. We selected fraction 3 for er vesicles and fraction 7 for the plasma membrane vesicles. (FIG. 17B) Nanoparticle tracking analysis for erMEVs and pmMEVs. (FIG. 17C) Atomic force microscopy (AFM) image of the single erMEV. (FIG. 17D) Information including height and diameter of the erMEV obtained from AFM. Size of the vesicle was found to be around 200 nm and height about 50 nm.

FIG. 18A-18F. MEVs delivery to M2 macrophage. (FIG. 18A) Fluorescence image of DiI labeled erMEVs (FIG. 18B) Targeting specificity of erMEVs and pmMEVs onto M2 macrophages (FIG. 18C-18F) Time dependent uptake of erMEVs by M2 macrophages.

FIG. 19A-19D. Reprogramming macrophage polarization with erMEVs and pmMEVs. A) Measurement of pro-inflammatory cytokines including IL-12 and IL-10 produced after erMEVs delivery to M2 macrophages. 50,000 M2 macrophages were incubated with erMEVs at different concentration. B) Measurement of pro-inflammatory cytokines including IL-12 and IL-10 produced after pmMEVs delivery to M2 macrophages. 50,000 M2 macrophages were incubated with pmMEVs at different concentration. C) Ratio of IL-12 to IL-10 released by er or pm treated MEVs at different concentration. D) Measurement of pro-inflammatory cytokines produced by M2 and M1 macrophages compared to the production of cytokines released after er and pmMEV delivery to M2 macrophages. M2 macrophages are polarized toward an M1 phenotype upon interaction with both erMEVs and pmMEVs in vitro. Each data point is the average of at least five experiments (n=5). The data are presented as the mean±SEM. Comparison by paired t-test. For all statistical comparisons: *p<0.05, **p<0.01, ***p<0.001.

FIG. 20A-20F. erMEVs and pmMEVs exhibit differential ability to shift co-cultured M2 macrophages to M1 phenotype. FIG. 20A, 20D) Measurement of cytokine TNF-α released by M2 macrophages co-cultured with cancer cells and MEVs. Supernatants were collected 24 hours after the addition of erMEVs or pmMEVs. erMEVs exhibit higher ability to shift M2 macrophages towards an M1 phenotype compared to pmMEVs under co-cultured conditions. FIG. 20B, 20E) erMEVs and pmMEVs exhibit cancer cell killing ability under co-culture conditions. erMEVs show a higher ability inhibit cancer cell growth compared to pmMEVs under co-culture condition. FIG. 20C, 20F) Assessment of cancer cell viability upon incubation with equal number of erMEVs and pmMEVs. 20,000 A549 or Caov3 cells were cultured alone or with mouse 40,000 M2 bone marrow derived macrophages and 1.2×10¹⁰ MEVs were added to them. Cells were incubated with the same number of erMEVs and pmMEVs for 72 hours. Cell viability was assessed at 72 hours post vesicles addition. The percent cell viability was obtained by comparing cells treated with erMEVs or pmMEVs respective to the untreated control. Each data point is the average of at least three experiments (n=3). The data are presented as the mean±SEM. Each data point is the average of at least five experiments (n=5). The data are presented as the mean±SEM. Comparison by one way ANNOVA. For all statistical comparisons: p<0.001.

FIG. 21A-21C. Identification of proteins present in erMEVs and pm MEVs that are likely contributing the repolarization. (FIG. 21A) Chemokine expression profile of erMEVs and pmMEVs determined by mouse chemokine antibody array and identification of possible chemokines that could alter macrophage phenotype. (FIG. 21B) Comparison of the mean pixel density measurement between chemokines of erMEVs and pmMEVs (FIG. 21C) Western blot analysis of different exosome protein markers and endogenous ligands present on erMEVs and pmMEVs. Equal amounts of total proteins extracted from M1 macrophages, erMEVs, and pmMEVs were immunoblotted for CD54, MHCII, CD63, CD9, HSP60, HSP90B1, CD40L, CD137L, CD95L, RANKL, TNFα, and OX40L.

FIG. 22A-22D. Antibody treatment of MEVs on macrophage reprogramming. (FIG. 22A) Antibody treatment of MEVs significantly decreases their ability to reprogram M2 to M1 phenotype. MEVs were treated with antibodies including HSP90B1 alone, OX40L alone, RANKL alone, and a cocktail of antibodies including HSP90B1, OX40L, RANKL, ICAM1, CD63, HSP60, and CCL5. TNF-α released by the MEV treated M2 macrophages were assayed using mouse TNF-α V-plex assay from Meso Scale Discovery. (FIG. 22B) Western blot analysis of SGK1 protein on erMEVs and pmMEVs. (FIG. 22C) SGK-1 antibody treatment of erMEVs significantly increases the production of TNF-α by MEV-treated M2 macrophages. (FIG. 22D) SGK-1 antibody treatment of erMEVs significantly increases the production of TNF-α by MEV-treated M2 macrophages. Each data point is the average of at least five experiments (n=5). The data are presented as the mean±SEM. Comparison by one way ANNOVA. For all statistical comparisons: *p<0.05, **p<0.01, ***p<0.001.

FIG. 23. In vivo delivery. Mice bearing A549 xenografts (FIG. 23A, 23C) were injected with DiR labeled erMEVs (FIG. 23B) and DiR labeled pmMEVs (FIG. 23D) respectively, demonstrating that both erMEVs and pmMEVs can reach the tumor of mice.

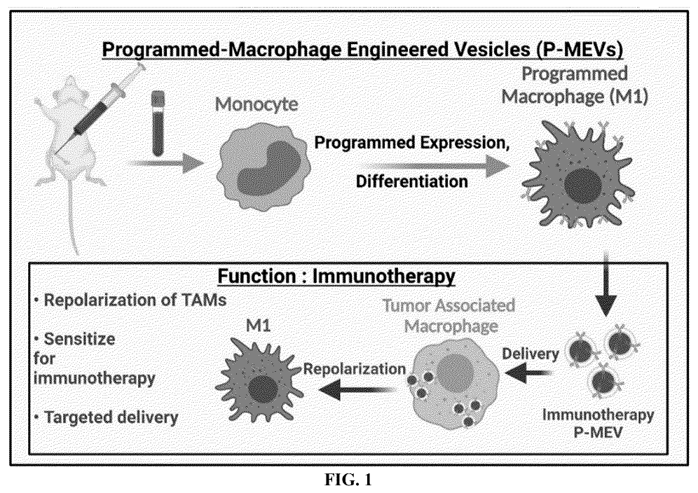

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes programmed cell-derived vesicles (CDVs), methods of making programmed CDVs, and methods of using programmed CDVs.

A CDV is a vesicle generated from the membrane of a cell. For example, a vesicle can be generated from a cell that is a tumor cell, a dendritic cell, a macrophage, or another type of cell.

In some embodiments, the method of making a programmed CDV comprises obtaining a cell from which the CDV will be generated (sometimes referred to herein as a donor cell or parent cell), fragmenting the membrane of the donor cell and allowing the fragmented membrane to assemble into a CDV. Prior to fragmentation, a ligand of interest can be overexpressed on the surface of the donor cell and/or the donor cell can be isolated from an organelle of interest. In this regard, the ligand of interest can be a protein of interest. When the donor cell is fragmented, the resulting fragmented membrane displays the ligand of interest and/or is organelle-specific. The fragmented membrane is placed in an assembly solution, where it assembles into the CDV. Accordingly, the resulting CDV displays the ligand of interest and/or is an organelle-specific CDV. In some embodiments, the method of making a programmed CDV comprises obtaining a donor cell, overexpressing a ligand of interest on the surface of the cell, and fragmenting the membrane of the cell and allowing the fragmented membrane to assemble into a CDV displaying the ligand of interest on its surface. In some embodiments, the method of making a programmed CDV comprises isolating a donor cell from an organelle of interest, and fragmenting the membrane of the cell and allowing the fragmented membrane to assemble into an organelle-specific CDV.

Fragmentation of the membrane of the cell can be achieved using a variety of methods known in the art. Such fragmentation methods can include, for example, sonication, such as by using ultrasonic probes or baths, detergent lysis, osmotic shock, freeze-thaw cycles, and mechanical disruption. Each of these methods has its own operational characteristics and potential drawbacks. For instance, techniques that generate high levels of heat, such as certain forms of sonication, can denature proteins and compromise the functionality of the resulting vesicles, making them less suitable for applications requiring intact membrane proteins or functional surface markers. Another example of a fragmentation method that can be used in accordance with the presently-disclosed subject matter is nitrogen cavitation. In some methods that make use of nitrogen cavitation, the cells are subjected to high-pressure nitrogen gas, e.g., between 200 and 500 psi, in a pre-chilled decompression chamber. The rapid release of this pressure causes the cell membranes to rupture and fragment, leading to the spontaneous formation of vesicles in solution. This method is useful due to its ability to produce relatively uniform vesicles without generating heat, which helps preserve protein integrity and avoids denaturing sensitive biomolecules.

As disclosed herein, in some embodiments, the donor cell is isolated from a organelle of interest, such that the CDV derived from that donor cell is an organelle-specific CDV. The organelle of interest can be, for example, endoplasmic reticulum (ER), plasma membrane (PM), or mitochondria. In some embodiments, the programmed CDV is an endoplasmic reticulum-derived macrophage-engineered vesicle (erMEV). In some embodiments, the programmed CDV is a plasma membrane-derived macrophage-engineered vesicle (pmMEV). In some embodiments, the programmed CDV is a mitochondria-derived macrophage-engineered vesicle.

As disclosed herein, in some embodiments, a ligand of interest is overexpressed on the surface of the donor cell, such that the CDV derived from that donor cell displays the ligand of interest on its surface. In some embodiments, the ligand of interest is overexpressed by transfecting the donor cell with a plasmid for expressing the ligand of interest.

In some embodiments, the ligand of interest is selected from the group consisting of Cluster of Differentiation 54 (CD54), also known as Intercellular Adhesion Molecule 1 (ICAM-1), Tumor Necrosis Factor-alpha (TNF-α), cytosine-phosphorothioate-guanine oligodeoxynucleotides (CpG-ODN), Inducible T-cell COStimulator (ICOS), and combinations thereof. In some embodiments, the programmed CDV displays CD54, TNF-α, CpG-ODN, ICOS, or combinations thereof on its surface.

In some embodiments, the ligand of interest is a polarization-inducing ligand. In some embodiments, the ligand of interest is a targeting-enhancing ligand. In some embodiments, the ligand of interest promotes cellular uptake of the CDV displaying the ligand of interest. In some embodiments, the ligand of interest targets a specific cell type and optionally modulates the specific target cell type. In some embodiments, the specific target cell type is an immune cell or a tumor cell. In some embodiments, wherein the ligand of interest has specific immunomodulatory properties, including the capability to bidirectionally modulate immune cell polarization. In some embodiments, the ligand of interest enhances cell reprogramming toward a proinflammatory phenotype.

In some embodiments, the programmed CDV displays a polarization-inducing ligand. In some embodiments, the programmed CDV displays a targeting-enhancing ligand. In some embodiments, the programmed CDV displays a ligand of interest that promotes cellular uptake in a target cell of the CDV displaying the ligand of interest. In some embodiments, the programmed CDV displays a ligand of interest that targets a specific cell type and optionally modulates the specific target cell type. In some embodiments, the programmed CDV is an MEV useful for re-polarizing M2 macrophages toward a proinflammatory phenotype.

As disclosed herein, in some embodiments, the donor cell can be a dendritic cell, a tumor cell, or a macrophage. For example, the donor cell can be a dendritic cell, which can be cultured and optionally stimulated to induce maturation and antigen presentation. For another example, the cell can be a tumor cell, such as a tumor cell taken from an in vivo environment, such as the site of a tumor in a subject, or tumor cell an in vitro environment, such as a cultured tumor cell line. The donor cell can also be a macrophage. In some cases, the macrophage can be a tumor-associated macrophage (TAM). In some cases, the macrophage can be a bone marrow-derived macrophage.

When a CDV is generated from a macrophage, it is sometimes referred to as a macrophage-engineered vesicle (MEV). Macrophages can be classified into three main phenotypes: M0, M1, and M2. M0 macrophages are considered unpolarized (M0 phenotype). M1 macrophages are associated with pro-inflammatory responses (M1 phenotype). M2 macrophages are anti-inflammatory (M2 phenotype).

Macrophages can be polarized toward a particular phenotype. For example, an M1 pro-inflammatory MEV can be generated from a donor macrophage that has been polarized to an M1 phenotype, e.g., from an M0 macrophage. Similarly, an M2 anti-inflammatory MEV can be generated from a donor macrophage that has been polarized to an M2 phenotype, e.g., from an M0 macrophage. Macrophages can also be repolarized to different phenotype. For example, an M1 macrophage can be repolarized to an M2 macrophage, which can then serve as a donor macrophage to generate an M2 anti-inflammatory MEV. Similarly, for example, an M2 macrophage can be repolarized to an M1 macrophage, which can then serve as a donor macrophage to generate an M1 pro-inflammatory MEV.

Macrophages can be polarized or repolarized using a variety of methods, for example, by exposure to specific cytokines, chemokines, cell surface proteins, receptor agonists, or engineered vesicles derived from other immune cells. For example, macrophages can be polarized by treating unstimulated (M0) macrophages with cytokines such as lipopolysaccharide (LPS) and/or interferon-gamma (IFN-γ) to induce a pro-inflammatory M1 phenotype, or with interleukin-4 (IL-4) and/or interleukin-13 (IL-13) to induce an anti-inflammatory M2 phenotype. Macrophages can also be repolarized, for example, through interaction with CDVs. For example, M1 MEV can be used to repolarize M2 macrophages toward a pro-inflammatory state. Similarly, M2 MEVs can be used to repolarize M1 macrophages towards an anti-inflammatory state. Additionally, as described herein, CDVs presenting certain ligands of interest can be useful for repolarizing macrophages. For example, CDVs presenting the cytokine TNF-α can be useful for repolarizing M2 macrophages to the M1 phenotype. For another example, CDVs presenting the chemokine CCL5 can be useful for activating M1 polarization and inhibiting M2 polarization. For another example, CDVs presenting ICAM-1 (also referred to as CD54) can be useful for inducing M1-like cytokine profiles in M2 macrophages. For another example, CDVs presenting CpG-ODN can be useful for repolarization to an M1 phenotype. For another example, CDVs presenting ICOS can be useful for repolarization.

Targeting a CDV to a desired site can be achieved using a variety of techniques. For example, administration routes can be selected to facilitate desired targeting of CDVs to specific target sites. For another example, targeting can be facilitated by donor cell specificity, i.e., vesicles derived from a particular cell type preferentially target and are taken up by the same cell type. For another example, targeting can be facilitated by leveraging phenotype-specific CDV properties, such as M1 phenotype or M2 phenotype. For another example, CDVs can have surface features derived their donor cell, which facilitate targeting. Such surface features can be inherent or naturally-occurring in the donor cell and/or they can be engineered. For example, donor cell can have inherent or naturally-occurring surface features that are maintained in the resulting CDV, preferentially directing the CDV to a target site. One example disclosed herein is in the case of an endoplasmic reticulum-derived MEV (erMEV) being targeted to the tumor microenvironment. For another example, a donor cell can be engineered such that it overexpresses a ligand of interest, and the resulting CDVs also overexpress the ligand of interest, preferentially directing the CDV to a target site. One example disclosed herein is in the case of CD54 being the ligand of interest, which interacts with receptors on target cells such as M2 macrophages, enhancing both targeting and repolarization.

In some embodiments of the presently-disclosed subject matter, the ligand of interest can be selected for interaction with a target of interest. As will be appreciated by one of ordinary skill in the art upon study of this document, the target of interest and ligand of interest pair can vary and/or be combined to facilitate the intended use of the CDV. For example, if there is a goal of impacting a condition, the target of interest can be the environment of the condition, such as the location of a wound or a tumor in a subject. For another example, when the goal is to deliver a therapeutic agent to target cells, the ligand of interest can be selected to bind a receptor expressed on the target cells, and the target of interest can be the target cell population within a in vivo environment, such as a tumor microenvironment or site of inflammation. The flexibility in pairing ligands and targets allows CDVs to be tailored for a wide range of biological and therapeutic applications.

As described hereinabove, it can be useful to polarize, repolarize, or shift the phenotype of a donor cell that is a macrophage before fragmenting the membrane of the macrophage and allowing the fragmented pieces to assemble into an MEV. Additionally, the presently-disclosed subject matter includes a method of shifting the phenotype of a target macrophage.

As described hereinabove, macrophages can be polarized or repolarized using a variety of methods. For example, macrophages can be repolarized through interaction with CDVs. For example, M1 MEV can be used to repolarize M2 macrophages toward a pro-inflammatory state. Similarly, M2 MEVs can be used to repolarize M1 macrophages towards an anti-inflammatory state. Additionally, as described herein, CDVs presenting certain ligands of interest can be useful for repolarizing macrophages. For example, CDVs presenting the cytokine TNF-α can be useful for repolarizing M2 macrophages to the M1 phenotype. For another example, CDVs presenting the chemokine CCL5 can be useful for activating M1 polarization and inhibiting M2 polarization. For another example, CDVs presenting ICAM-1 (also referred to as CD54) can be useful for inducing M1-like cytokine profiles in M2 macrophages. For another example, CDVs presenting CpG-ODN can be useful for repolarization to an M1 phenotype. For another example, CDVs presenting ICOS can be useful for repolarization.

The presently-disclosed subject matter includes a method of shifting a target macrophage phenotype to an M1 macrophage, which comprises contacting the target macrophage with an of endoplasmic reticulum-derived macrophage-engineered vesicle (erMEV). In some embodiments, the target macrophage is in an in vivo environment. In some embodiments, the in vivo environment is the site of a cancer in a subject.

As will be appreciated by the skilled artisan upon study of this document, M1 is a pro-inflammatory phenotype having utility in the context of cancer treatment, for example for use in sensitizing cancer to immunotherapy. Accordingly, shifting a macrophages in a in vivo environment at the site of a cancer can have an anti-cancer therapeutic effects. Meanwhile, M2 is an anti-inflammatory phenotype having utility in the context of conditions characterized by inflammation and for reducing potential toxic effects of the pro-inflammatory phenotype. Accordingly, shifting a macrophage in an in vivo environment at the site of a wound, an inflammatory disease, an infectious disease, a traumatic injury, or an ischemic event, or condition of the central nervous system can have a therapeutic effect.

As used herein, the terms treatment and treating relate to ameliorating at least one symptom of the condition and are inclusive of prophylactic treatment and therapeutic treatment. As would be recognized by one or ordinary skill in the art, treatment that is administered prior to clinical manifestation of a condition then the treatment is prophylactic (i.e., it protects the subject against developing the condition). If the treatment is administered after manifestation of the condition, the treatment can be therapeutic (i.e., it is intended to diminish, ameliorate, control, or maintain the existing condition and/or side effects associated with the condition). As will be recognized by one of ordinary skill in the art, terms such as suppress and inhibit do not refer to a complete elimination of a condition or symptoms thereof in all cases. Rather, the skilled artisan will understand that such terms refer to a reduction or decrease in a condition or symptom thereof. Such reduction or decrease can be determined relative to a control. In some embodiments, the reduction or decrease relative to a control can be about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease.

The presently-disclosed subject matter includes a method of shifting a target macrophage phenotype to an M1 macrophage, which comprises contacting the target macrophage with an of endoplasmic reticulum-derived macrophage-engineered vesicle (erMEV). In some embodiments, the target macrophage is in an in vivo environment. In some embodiments, the in vivo environment is the site of a cancer in a subject.

The presently-disclosed subject matter further includes a method of treating a cancer, which comprises contacting a target macrophage in a tumor environment with an erMEV. In some embodiments, the target macrophage is in an in vivo environment. In some embodiments, the in vivo environment is the site of a cancer in a subject.

The presently-disclosed subject matter further includes a method of shifting a target macrophage phenotype to an M1 macrophage, which comprises contacting the target macrophage with a CDV, such as an MEV, displaying a ligand of interest, selected from the group consisting of CD54, TNF-α, CpG-ODN, ICOS, and combinations thereof, and/or contacting the target macrophage with an MEV derived from an M1 macrophage. In some embodiments, the target macrophage is in an in vivo environment. In some embodiments, the in vivo environment is the site of a cancer in a subject.

The presently-disclosed subject matter further includes a method treating a cancer, comprising contacting a target macrophage in a tumor environment with a CDV, such as an MEV, displaying a ligand of interest, selected from the group consisting of CD54, TNF-α, CpG-ODN, ICOS, and combinations thereof, and/or contacting the target macrophage with an MEV derived from an M1 macrophage. In some embodiments, the target macrophage is in an in vivo environment. In some embodiments, the in vivo environment is the site of a cancer in a subject.

The presently-disclosed subject matter further includes a method of shifting a target macrophage phenotype to an M2 macrophage, which comprises contacting the target environment with a CDV, such as an MEV, derived from an M2 macrophage. In some embodiments, the target macrophage is in an in vivo environment. In some embodiments, the in vivo environment is the site of a condition of the central nervous system, a wound, an inflammatory disease, an infectious disease, a traumatic injury, or an ischemic event.

The presently-disclosed subject matter further includes a method of treating a condition of the central nervous system, a wound, an inflammatory disease, an infectious disease, a traumatic injury, or an ischemic event, which comprises contacting the target environment with a CDV, such as an MEV, derived from an M2 macrophage. In some embodiments, the target macrophage is in an in vivo environment. In some embodiments, the in vivo environment is the site of the condition of the central nervous system, a wound, an inflammatory disease, an infectious disease, a traumatic injury, or an ischemic event.

In some embodiments of the presently-disclosed subject matter, cargo is encapsulated within the CDV. As described herein, when making a CDV, the membrane of the donor cell is fragmented, and the fragmented membrane is placed in an assembly solution, where the cargo assembles into the CDV. When cargo is desired to be encapsulated within the CDV, the assembly solution can comprise the cargo, such that, when the fragmented membrane assembles into the CDV, the CDV encapsulates the cargo.

As will be appreciated by one of ordinary skill in the art upon study of this document, the cargo can be selected to facilitate the intended use of the CDV. For example, when the intended use is to elicit a physiological or cellular response upon delivery to a biological target, the cargo can be biologically-active cargo. Examples of cargo that can be encapsulated in accordance with the presently-disclosed subject matter include, but are not limited to, dyes, small molecules (chemical compounds less than about 1000 daltons), nucleotides, polypeptides or proteins, gene editing components, and other therapeutic or diagnostic agents. Examples of dyes include fluorescent markers useful for imaging or tracking vesicle delivery. Examples of small molecule drugs include, but are not limited to, chemotherapeutic and anti-tumor agents, as well as agents useful for treating conditions of the central nervous system, a wound, an inflammatory disease, an infectious disease, a traumatic injury, or an ischemic event. Examples of gene editing components include CRISPR-Cas9 systems and related nucleases. Examples of proteins include cytokines, enzymes, and antibodies. Additional examples include nanobodies, aptamers, engineered proteins, molecularly imprinted polymers, and scaffold proteins, all of which can be used for targeted binding or modulation of cellular pathways.

As will be appreciated by one of ordinary skill in the art upon study of this document, when the intended use of the CDV has a biological or therapeutic component, it can be beneficial to select both cargo and CDV features (e.g., ligand of interest, organelle of interest, phenotype) to align with and facilitate the biological or therapeutic goals. For example, when there is an anti-cancer goal, it can be beneficial to select an anti-cancer agent as cargo and selecting a CDV polarized to an M1-polarized macrophages (pro-inflammatory). For another example, the goal is to reduce inflammation or promote tissue repair, such as in the context of spinal cord injury or stroke, it can be useful to encapsulate anti-inflammatory agents and use CDVs derived from M2-polarized macrophages (anti-inflammatory). Similarly, when the goal is to stimulate an immune response, such as in vaccine delivery or immunotherapy, CDVs can be loaded with immunostimulatory cytokines or antigens and derived from dendritic cells or M1 macrophages. For another example, in gene therapy applications, CDVs can encapsulate plasmid DNA, mRNA, or gene editing components such as CRISPR-Cas9. For another example, in diagnostic or imaging contexts, CDVs can encapsulate fluorescent dyes or contrast agents. In another cases, the CDV surface can also be engineered to display targeting ligands or immune-modulatory proteins, while the interior can encapsulate biologically active cargo such as enzymes, cytokines, or small molecule drugs. Accordingly, the combination of surface and internal features can be tailored to enhance delivery, targeting, and functional outcomes.

As disclosed herein, the presently-disclosed subject matter further includes cell-derived vesicles (CDV). In some embodiments, the CDV is prepared by a method as disclosed herein.

In some embodiments, the CDV comprises a membrane from a donor cell overexpressing a ligand of interest, such that the CDV displays the ligand of interest on its surface. In some embodiments, the donor cell is transfected with a plasmid for expressing the ligand of interest. In some embodiments, the ligand of interest is selected from the group consisting of CD54, TNF-α, CpG-ODN, ICOS, and combinations thereof. In some embodiments, the ligand of interest is a polarization-inducing ligand and/or a targeting-enhancing ligand. In some embodiments, the ligand of interest targets a specific cell type and optionally modulates the specific target cell type. In some embodiments, the specific target cell type is an immune cell or a tumor cell. In some embodiments, the ligand of interest has specific immunomodulatory properties, including the capability to bidirectionally modulate immune cell polarization. In some embodiments, the ligand of interest enhances cell reprogramming toward a proinflammatory phenotype. In some embodiments, the CDV is an MEV useful for re-polarizing M2 macrophages toward a proinflammatory phenotype. In some embodiments, the ligand of interest promotes cellular uptake of the CDV displaying the ligand of interest. In some embodiments, the CDV is polarized to an M1 phenotype. In some embodiments, the CDV is polarized to an M2 phenotype. In some embodiments, the ligand of interest is selected for interaction with a target of interest. In some embodiments, the ligand of interest selectively binds the target of interest. In some embodiments, the target of interest is on a target cell. In some embodiments, the target of interest is in an in vivo environment. In some embodiments, the ligand of interest is selected from the group consisting of CD54, TNF-α, CpG-ODN, ICOS, and combinations thereof. In some embodiments, the CDV further comprises cargo encapsulated within the CDV. In some embodiments, the cargo is a therapeutic agent and/or a diagnostic agents. In some embodiments, the cargo is a dye, small molecule, nucleotide, polypeptide, a gene editing component, or combinations thereof.

In some embodiments, the CDV comprises a membrane from a donor cell isolated from an organelle of interest, which that the CDV is organelle-specific. In some embodiments, the organelle of interest is selected from the group consisting of endoplasmic reticulum (ER), plasma membrane (PM), mitochondria. In some embodiments, the CDV is an is an endoplasmic reticulum-derived macrophage-engineered vesicle (erMEV), a plasma membrane-derived macrophage-engineered vesicle (pmMEV), or a mitochondria-derived macrophage-engineered vesicle. In some embodiments, a surface feature of the donor cell interacts with a target of interest. In some embodiments, the surface feature selectively binds the target of interest. In some embodiments, the target of interest is on a target cell. In some embodiments, the target of interest is in an in vivo environment. In some embodiments, the donor cell is isolated from endoplasmic reticulum. In some embodiments, the CDV is an endoplasmic reticulum-derived macrophage-engineered vesicle (erMEV). In some embodiments, the CDV further comprises cargo encapsulated within the CDV. In some embodiments, the cargo is a therapeutic agent and/or a diagnostic agents. In some embodiments, the cargo is a dye, small molecule, nucleotide, polypeptide, a gene editing component, or combinations thereof.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, in some embodiments±0.1%, in some embodiments±0.01%, and in some embodiments±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

### Example 1: Macrophage-Engineered Vesicles (MEVs) Characterization

Pro-inflammatory (M1) bone marrow-derived macrophages (BMDMs) were used to generate macrophage-engineered vesicles (MEVs) through the disruption of the cell membrane with nitrogen cavitation. This leads to the formation of nano-sized membrane fragments that rearrange to form vesicles.

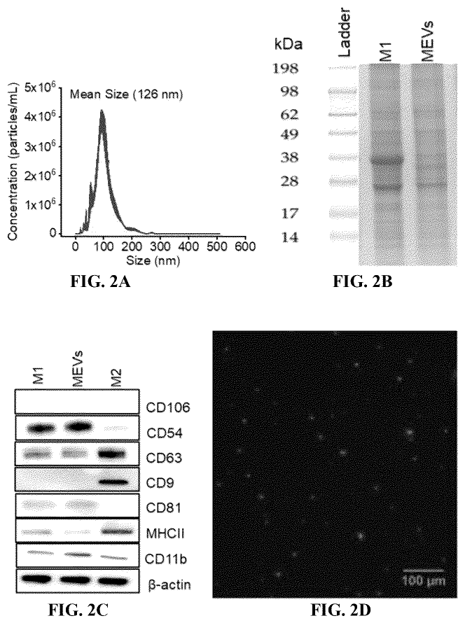

To characterize vesicle concentration and size distribution, nanoparticle tracking analysis (NTA) was used, which allows for measuring both the size distribution and the concentration of the vesicles in solution. 100 million M1 BMDMs generated≈2×1012 MEVs. The size distribution of the MEVs obtained from nanoparticle tracking analysis is primarily between 50-200 nm (FIG. 2A), which is similar to the range reported for exosomes. The mean diameter of MEVs obtained from NTA was 127 nm.

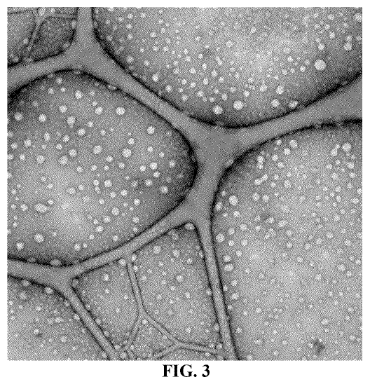

Transmission electron microscopy (TEM) was also performed, which gave similar results in terms of vesicle diameter and size distribution (FIG. 3). To determine the stability of MEVs, the surface charge of the MEVs was measured using a Malvern Zetasizer. The zeta potential of MEVs was −5.2 mV. This is similar to values seen for endogenously released vesicles such as exosomes which have been reported to have a zeta potential between −5 to −20 mV.

Sodium dodecyl sulfate-polyacrylamide gel electrophoresis (SDS-PAGE) was used to determine the proteins present on the surface of MEVs in comparison to the parent M1 macrophage. Similar protein bands were observed from MEVs and M1 macrophages, indicating that vesicle formation retains most of the same proteins present on the cell surface (FIG. 2B).

Exosomal marker proteins such as tetraspanins (CD9 and CD63), integrins (CD81, CD82), chaperones heat shock proteins 60 and 70 (HSP60, HSP70), immunoglobulins (intracellular adhesion molecule 1 (ICAM-1) and vascular adhesion protein 1 (VCAM1)), and major histocompatibility complex class I (MHC-I) and II (MHC-II) have been shown to be present on M1 exosomes and have been implicated in adhesion, signaling, and activation when exosomes are delivered to target macrophages. Western blotting was performed for several of these exosomal marker proteins including CD9, CD54, CD63, CD81, and CD106 and the majority of these proteins including CD54 (ICAM-1), CD63, MHCII, CD11b, CD81 are present in MEVs (FIG. 2C). However, other exosomal marker proteins including CD9, and CD106 were absent in MEVs. Western blotting analysis demonstrated that CD9 and CD106 were also absent from parent M1 BMDMs.

Tetraspanins including CD9, and CD63 are integral membrane proteins, embedded within the cellular membranes and have been shown to play a vital role in the fusion of exosomes with target cells. Similarly, ICAM-1 is a transmembrane glycoprotein that has been shown to mediate cell-cell interaction and outside-in cell signaling during an immune response. The presence of a majority of the same surface markers in MEVs indicates that vesicles engineered through nitrogen cavitation likely have similar properties to exosomes.

A set of experiments were performed to determine whether MEVs could successfully be delivered to M2 BMDMs, similar to what has been shown for exosomes. MEVs were labeled with 1,1′-dioctadecyl-3,3,3′,3′-tetramethylindocarbocyanine perchlorate (DiI), a lipophilic, non-toxic, fluorescent label that embeds into the membrane of the vesicles. The labeling of these vesicles was confirmed using fluorescence microscopy. The red punctate regions seen in FIG. 2D indicate the successful labeling of vesicles with DiI.

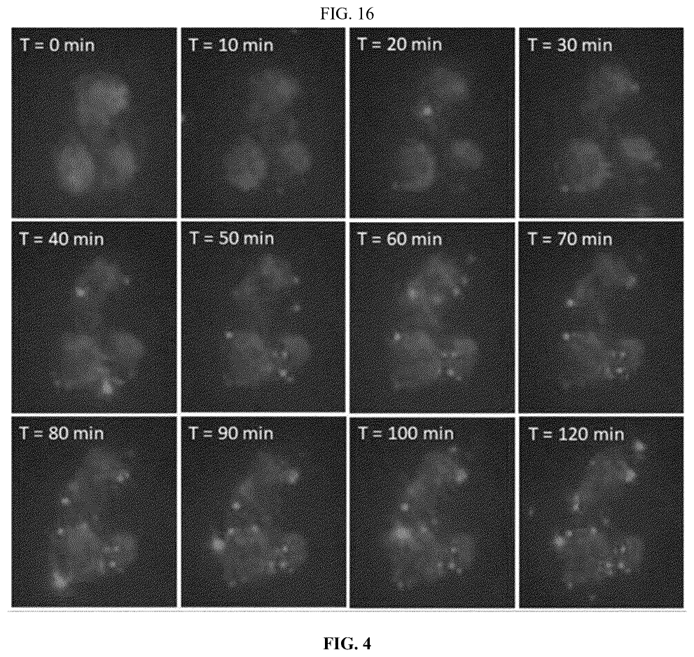

These labeled vesicles were used in time-lapse imaging experiments to record vesicle delivery at multiple time points over the course of 2 h by incubating M2 BMDMs with 1×10⁹ labeled vesicles. MEV uptake was tracked using the fluorescence intensity of the labeled vesicles in the otherwise unlabeled cells. Images were taken at 10-minute time intervals for 2 h. A gradual uptake of MEVs by M2 BMDMs was observed over time, as indicated by the increase in fluorescence intensity (FIG. 4).

These characterization results suggest that nitrogen cavitation-generated MEVs have similar size, zeta potential, and surface markers as exosomes and these vesicles are efficiently taken up by M2 BMDMs.

### Example 2: MEV-Mediated Phenotypic Reprogramming of M2-BMDMs

The ability of pro-inflammatory (M1) MEVs to reprogram anti-inflammatory (M2) BMDMs toward a proinflammatory phenotype was confirmed using a series of cell membrane modifications. A shift in macrophage phenotype was validated using immunocytochemistry analysis to measure the expression of inducible nitric oxide synthase (iNOS), a pro-inflammatory macrophage marker, in M2 BMDMs after they were incubated with different concentrations of MEVs derived from M1 cells.

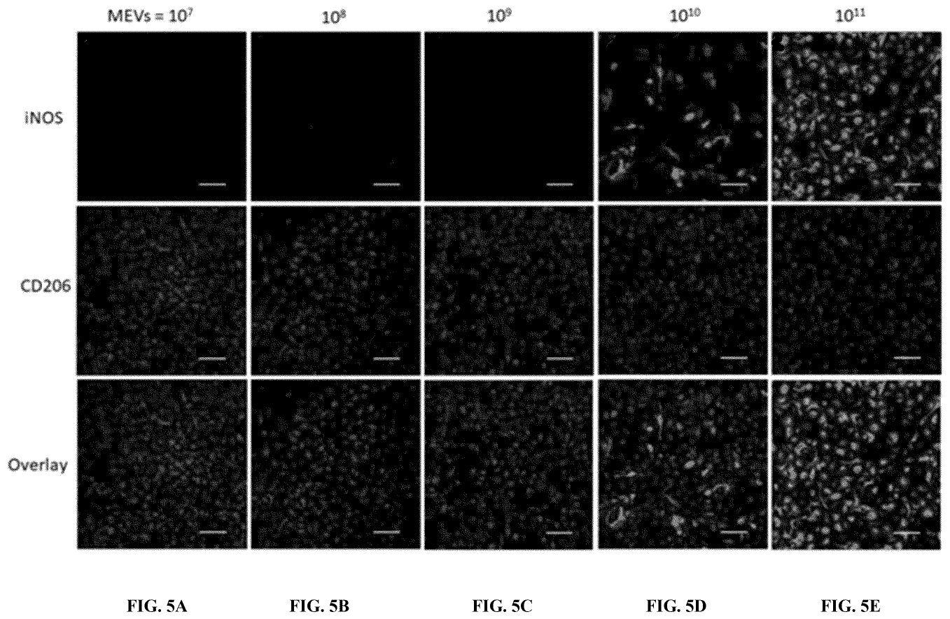

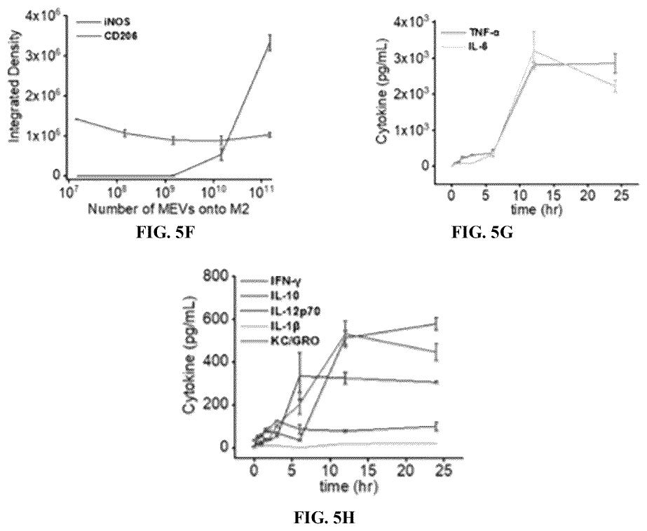

50 000 M2 BMDMs were incubated with increasing concentrations of MEVs ranging from 107 to 1011 for 12 h at 37° C. A clear increase in iNOS was observed for M2 BMDMs upon incubation with increasing concentrations of MEVs (FIG. 5A-5E). At a concentration of 1011 vesicles, a robust expression of iNOS was observed (FIG. 5E). These results demonstrate that M2 BMDMs can be reprogrammed through MEV exposure, shifting polarization toward a pro-inflammatory phenotype.

Additional immunocytochemistry analysis was performed to understand the effect of MEV-driven M2 to M1-like polarization on the expression of CD206 an M2 macrophage marker. A gradual decrease in CD206 expression was observed (FIG. 5A-5F). Overall, these results indicate that at a suitable concentration, MEVs can reprogram M2 BMDMs toward an M1-like phenotype.

A time-dependent repolarization assay was also performed for M2 macrophages incubated with MEVs and simultaneously assessed seven different pro-inflammatory cytokines (IFN-γ, IL-10, IL-12p70, IL-1β, IL-6, KC/GRO, and TNF-α) released by MEV-treated M2BMDMs. 1×10¹¹ vesicles were added to 50 000 macrophages in culture and analyzed cytokine release by MEV-treated M2 macrophages after 0.5, 1, 1.5, 2, 3, 6, 12, and 24 h of incubation.

Compared to the low level of pro-inflammatory cytokines released by unmodified M2 BMDMs, an increase in cytokine levels was observed as early as 2 h of incubation. As the incubation time progresses, an increase in cytokine release by MEV-treated M2 BMDMs was observed. Pro-inflammatory cytokine releases into the supernatant plateaued after 12 h of incubation of M2 BMDMs with MEVs (FIG. 5G-5H). Differences in the cytokine release by MEV-treated M2 BMDMs were not observed between 12 and 24 h of incubation time. These results indicate that MEV-mediated M2 to M1 repolarization depends both on the concentration of MEVs and the incubation period.

### Example 3: Role of MEV-Anchored Endogenous Ligands on M2 to M1 Macrophage Modulation

The present inventors contemplated that proteins anchored within the membrane of MEVs control targeting, cellular uptake, and drive changes in macrophage phenotype. Because MEVs are generated from parent pro-inflammatory macrophages and maintain proteins resident on the surface of M1 macrophages (FIG. 2B), MEVs may carry a wide range of membrane-bound cytokines, chemokines, transmembrane proteins, and other cell signaling endogenous ligands that belong to the parent macrophage. These proteins likely interact with the surface proteins on the recipient anti-inflammatory (M2) macrophages initiating signaling cascades that lead to their repolarization toward an M1 phenotype.

A set of studies was performed to validate that membrane proteins played a role in MEV-induced macrophage repolarization. It is possible that cytokines, cell-associated signaling proteins, anchored in the plasma membrane of the cell could be responsible for macrophage reprogramming. For example, pro-inflammatory cytokines including TNF-α, IFN-γ, and IL-12 produced by classically activated macrophages can stimulate macrophage polarization toward an M1 phenotype.

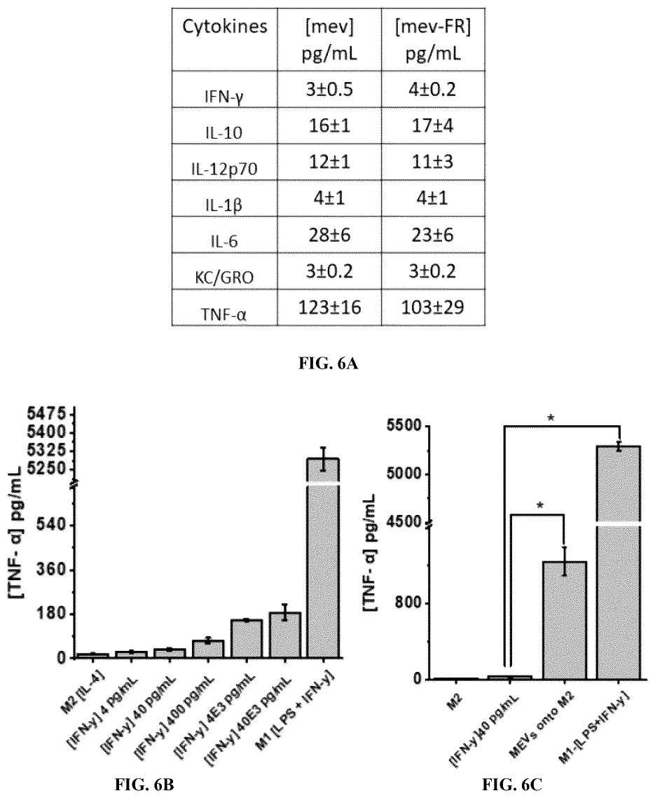

Vesicle solutions were examined for the presence of seven pro-inflammatory cytokines including IFN-y, IL-10, IL-12p70, IL-1β, IL-6, KC/GRO, and TNF-α to determine if residual cytokines contributed to MEV-mediated M2 to M1 repolarization. To analyze the cytokines entrapped in the interior of MEVs, MEVs were freeze-fractured to rupture them and release the entrapped cargo from inside of the vesicles. Cytokine assays showed that MEVs contain low levels of pro-inflammatory cytokines in the interior of the vesicles and virtually no cytokines in the vesicle solution (FIG. 6A).

Further studies were conducted to determine whether the concentration of cytokines present in the vesicle suspension was sufficient to reprogram M2 macrophages toward an M1 phenotype. Following an IFN-y dose response with M2 macrophages, tumor necrosis factor alpha (TNF-α) released by M2 macrophages into the supernatant after treatment was assessed (FIG. 6C). Results showed that the small amount of cytokines present in MEVs (˜40 pg mL-1) was not capable of altering the M2 macrophage phenotype and had a negligible contribution to mediating M2 to M1 repolarization (FIG. 6B).

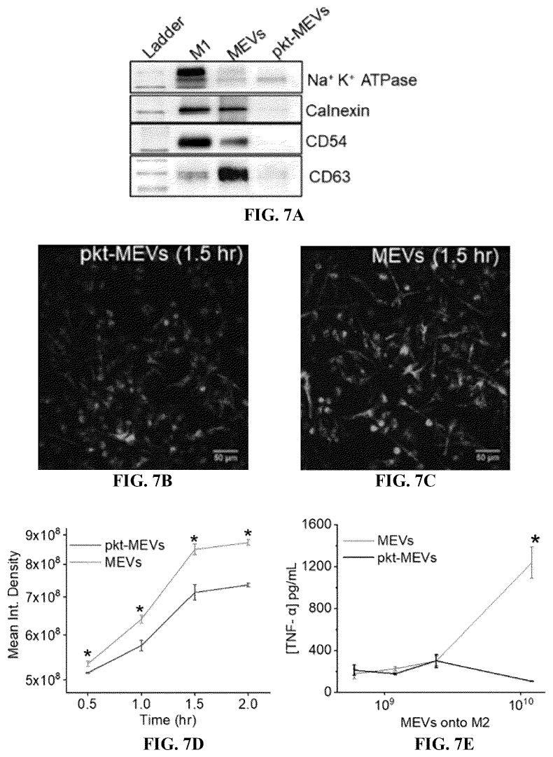

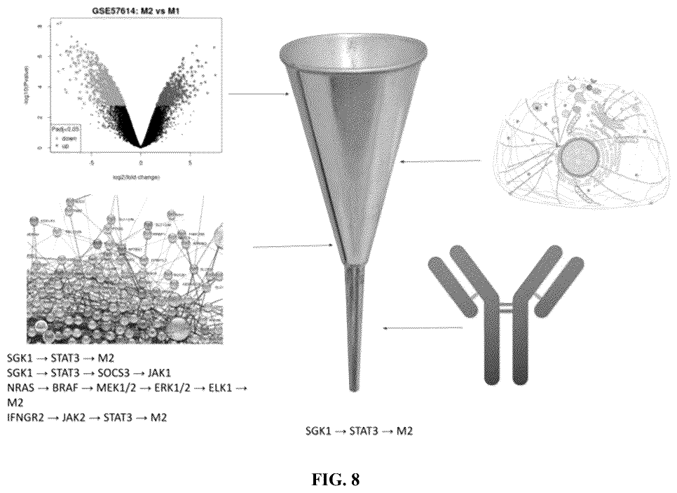

Additional studies were conducted to determine the importance of ligand-receptor interactions on the uptake of MEVs by M2 macrophages and MEV-mediated M2 to M1 macrophage repolarization. For this, proteolytic digestion of the membrane proteins embedded in the membrane of MEVs was carried out using proteinase-K (0.5 mg mL-1). Proteinase-K is a broad-spectrum proteolytic enzyme that is commonly used to digest proteins. A western blotting analysis was performed to confirm the elimination of membrane-anchored proteins present on the surface of MEVs after proteinase-k digestion. The expression of Na₊K₊ ATPase (a plasma membrane marker), calnexin (an endoplasmic reticulum marker), CD54 (a transmembrane glycoprotein), and CD63 (a transmembrane protein) in M1 macrophages (control), MEVs, and proteinase-K-treated MEVs were compared. Proteinase-K treatment of MEVs resulted in nearly the complete digestion of the membrane proteins that were analyzed (FIG. 7A).

Next, a set of experiments were performed to compare the delivery of purified proteinase-K treated MEVs (pkt-MEVs) and regular untreated MEVs to target M2 macrophages. MEVs were labeled with the lipophilic fluorescent dye, DiI, and further purified from free dye before incubation with M2 macrophages. Equal numbers of DiI-labeled pkt-MEVs and DiI-labeled MEVs were left to incubate with separate M2 macrophage cultures for 0.5, 1, 1.5, and 2 h. Wide-field microscopy was used to compare the uptake of fluorescently labeled pkt-MEVs by M2 BMDMs. Proteinase-K treatment of MEVs resulted in a 20% reduction in the uptake of MEVs by M2 macrophages (FIG. 7B-7D). Even after the digestion of membrane proteins present on MEVs, the limited loss of cellular uptake of pkt-MEVs compared to untreated MEVs indicates that MEV uptake by M2 macrophages is not solely dependent on ligand-receptor interaction but likely driven by the inherent phagocytotic ability of M2 BMDMs.

Next, studies were conducted to test the ability of proteinase K-treated MEVs (pkt-MEVs) to reprogram M2 macrophages toward an M1 phenotype. M2 macrophages were incubated with different concentrations of pkt-MEVs and an assessment was made of the release of the pro-inflammatory cytokine TNF-α secreted by M2 macrophages compared to TNF-α secreted by M2 macrophages that had been incubated with the corresponding concentration of undigested MEVs. Eliminating the proteins on the surface of MEVs resulted in a near-complete loss in the ability of MEVs to re-polarize M2 BMDMs toward an M1 phenotype (FIG. 7E). This suggests that interactions between the membrane proteins present in MEVs, and the surface proteins present in M2 macrophages are the primary driver of MEV-induced macrophage repolarization.

### Example 4: Programming Nanovesicles With Endogenous Ligands for Enhanced Macrophage Repolarization

In the previous section, membrane proteins anchored on MEVs were shown to play a vital role in MEV-mediated M2 to M1 polarization. To identify membrane proteins that might enhance macrophage polarization in response to MEV delivery a computational pathway analysis protocol was used from Talley, et al., (2022) (FIG. 8). For this, the present inventors contemplated that M1-derived MEVs contain significantly higher expression levels of proteins that support or suppress pathways associated with M1 versus M2 polarization. This was tested using messenger ribonucleic acid (mRNA) data from National Center for Biotechnology Information (NCBI) GSE57614 that was found to be consistent with the data in FIG. 5A-5H to first identify differentially expressed genes (DEGs) in M1 macrophages.

TABLE 1
Differentially expressed genes in M1 macrophage. The logFC was
calculated by log(M1)-log(M2). In this case, positive logFC
indicate upregulation of genes such as TNF, IL6 and IL1B in
M1 and downregulation of genes CD36, ADORA3 and TGFBR2 in M1.

| Gene Symbol | LogFC | Gene Symbol | LogFC |
|---|---|---|---|
| TNF | 2.05 | CD36 | −5.22 |
| IL6 | 4.77 | ADORA3 | −5.51 |
| CXCL1 (KC/GRO) | 6.14 | TGFBR2 | −3.77 |
| IL1B | 6 | PRKCA | −3.76 |
| IFNG | 4.25 | CSF1R | −3.04 |
| CCL5 | 5.85 | CD9 | −2.9 |
| CD54 (ICAM1) | 4.95 | TLR5 | −4.86 |
| TLR2 | 2.41 | IL1R1 | −3.91 |
| TNFSF13B | 2.04 | CD206 (MRC1) | −4.06 |
| CD63 (LAMP3) | 6.41 | GPR34 | −5.73 |
| CD40 | 1.67 | FFAR4 | −2.19 |
| CD95 (FAS) | 4.76 | GNAQ | −2.55 |
| CXCL9 | 7.63 | CD180 | −5.15 |
| CCL1 | 3.52 | ALOX15 | −4.19 |
| CXCL2 (MIP-2) | 3.23 | CCL26 | −5.21 |

Log FC (defined as log(M1/M2)) was calculated in Table 1, where positive log FC indicates upregulation of genes for TNFα, IL6, and ILIB and downregulation of genes for CD36, ADORA3, and TGFBR2 in M1 macrophages. The log FC values shown in Table 1 for transcripts IL6, IL1β, TNF-α, CCL5, and ICAM1 indicated their upregulation whereas transcripts representative of anti-inflammatory phenotype include TGFBR2, ADORA3, FFAR4 are downregulated. Log FC values obtained from the computational approach were in fact representative of the experimental results. CD54 (ICAM1) is elevated in M1 and MEVs and CD9 is downregulated in M1 macrophages and MEVs relative to M2-polarized macrophages (FIG. 2C). In addition, the experimental results are consistent with the RNA data, as CD54 (ICAM1) is up-regulated and CD9 is down-regulated in M1 phenotypes. Additionally, CD206 (MRC1), a known M2 macrophage marker, is downregulated in the RNA data.

The genes identified above were cross-referenced against the manually curated database of macrophage signaling pathways. This database was represented by a network whose nodes corresponded to genes or expressed proteins, with interactions shown by directed edges. The edges were weighted to reflect a) whether the upstream node activates or inhibits the downstream node, b) if the node is substantiated by mRNA transcripts, and c) if the node is confirmed by mAb. A search was conducted for paths in the network that agonized M1-associated pro-versus anti-inflammatory phenotypes for each gene localized to the plasma membrane. Candidate plasma-membrane receptors and their corresponding ligands driving pro-inflammatory pathways that were identified by the approach are listed in Table 2. The highest-ranked pathways include the TNF and interleukin-1 receptors, which activate pathways well-known to polarize macrophages into pro-inflammatory states. Based on these results, TNF-α was predicted as one of the possible targets for overexpression in MEVs aiming to achieve MEVs with the highest likelihood of polarizing target macrophages.

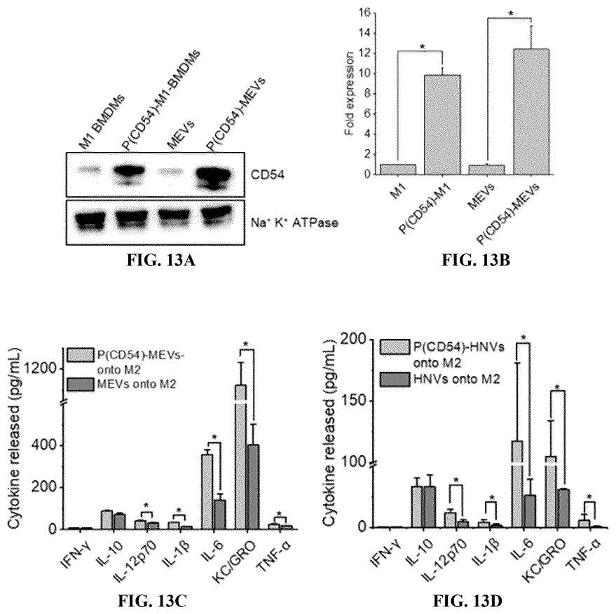

TABLE 2
M1 polarization pathways and their associated membrane
receptors. Pathways are shown in FIG. 13 for clarity.

| Receptor | Score | Signaling pathway | Ligand |
|---|---|---|---|
| TLR2 | 5.75 | ‘TLR2’, ‘RAC1’, ‘PI3K’, | PAMPs |
| | | ‘PDPK1’, ‘AKT’, ‘NFKB1’, | |
| | | ‘IL1B’, ‘M1 polarization’ | |
| CCR3 | 6 | ‘CCR3’, ‘GNAI1', ‘PI3K’, | CCL5 |
| | | ‘PDPK1’, ‘AKT’, ‘NFKB1’, | |
| | | ‘IL1B’, ‘M1 polarization’ | |
| TLR4 | 6 | ‘TLR4’, ‘TICAM2’, ‘TICAM1’, | PAMPs |
| | | ‘TBK1’, ‘IKBKE’, ‘IRF3’, ‘M1 | |
| | | polarization’ | |
| CCR1 | 6 | ‘CCR1’, ‘GNAI1’, ‘PI3K’, | CCL3, CCL5 |
| | | ‘PDPK1’, ‘AKT’, ‘NFKB1’, | |
| | | ‘IL1B’, ‘M1 polarization’ | |
| CCR5 | 6 | ‘CCR5’, ‘GNAI1’, ‘PI3K’, | CCL3, CCL4, |
| | | ‘PDPK1’, ‘AKT’, ‘NFKB1’, | CCL5 |
| | | ‘IL1B’, ‘M1 polarization’ | |
| TLR1 | 6 | ‘TLR1’, ‘RAC1’, ‘PI3K’, | PAMPs |
| | | ‘PDPK1’, ‘AKT’, ‘NFKB1’, | |
| | | ‘IL1B’, ‘M1 polarization’ | |
| TNFRSF1B | 7 | ‘TNFRSF1B’, ‘TRAF2’, ‘RIPK1’, | TNF-α |
| | | ‘MAP3K1’, ‘MAP2K7’, | |
| | | ‘MAPK8’, ‘IRF3’, ‘M1 | |
| | | polarization’ | |
| ITGAL | 7 | ‘ITGAL’, ‘PTK2B’, ‘RAC1’, | ICAM1 |
| | | ‘PI3K’, ‘PDPK1’, ‘AKT’, | |
| | | ‘NFKB1’, ‘IL1B’, ‘M1 | |
| ITGB2 | 7 | ‘ITGB2’, ‘PTK2B’, ‘RAC1’, | ICAM1 |
| | | ‘PI3K’, ‘PDPK1’, ‘AKT’, | |
| | | ‘NFKB1’, ‘IL1B’, ‘M1 | |
| TNFRSF1A | 7.5 | ‘TNFRSF1A’, ‘TRADD’, | TNF-α |
| | | ‘FADD’, ‘RIPK1’, ‘MAP3K1’, | |
| | | ‘MAP2K7’, ‘MAPK8’, ‘IRF3’, | |
| | | ‘M1 polarization’ | |

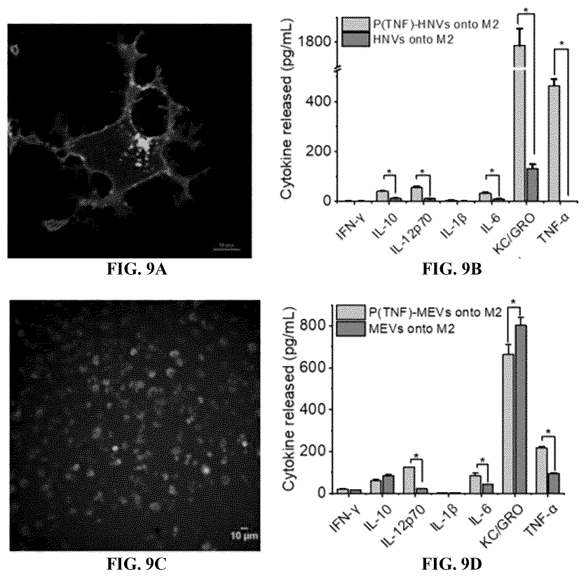

To validate the strategy, TNF-α was overexpressed on the cell membrane and TNF-α overexpressing was programmed to modulate macrophage polarization. In order to program cells by over-expressing TNF-α on their membrane, HEK cells were transfected, then proceed to mouse bone marrow-derived macrophages. A green fluorescent protein (GFP)-tagged mouse TNF-α plasmid was used for transfection to identify expression. TNF-α expressing HEK cells were used to generate programmed HEK cell-derived nanovesicles P(TNF)-HNVs. TNF-α expressing BMDMs were polarized to an M1 phenotype using LPS+IFN-γ and those cells were used for generating programmed macrophage-engineered vesicles (P(TNF)-MEVs). TNF-α overexpression was confirmed on the cells using confocal microscopy, which showed clear GFP fluorescence on the surface of these programmed cells (FIG. 9A, 9C). The macrophage repolarization efficacy of programmed nanovesicles including P(TNF)-HNVs and P(TNF)-MEVs were compared relative to their respective controls, using equal concentrations of vesicles in the experimental and control groups. The repolarization efficacy of the programmed nanovesicles was evaluated based on the cytokine production of nanovesicle-treated M2 BMDMs. Incubating M2 BMDMs with programmed HEK cell-derived nanovesicles P(TNF)-HNVs resulted in high levels of pro-inflammatory cytokines such as IL-12p70, IL-6, IL-10, KC/GRO, and TNF-α (FIG. 9B). However, unmodified HEK cell-derived vesicles elicit virtually no proinflammatory cytokine production in M2 BMDMs.

Consistent with the computational pathways analysis approach, M2 macrophages treated with programmed MEVs P(TNF)-MEVs produce significantly higher levels of pro-inflammatory cytokines such as Il-12. These results indicate that, under similar conditions, programmed nanovesicles exhibit greater immunomodulatory properties compared to unmodified cell-derived vesicles illustrating the capability to program functionality into MEVs via protein expression.

The computational pathway analysis approach indicated that the interaction of TNFSR plasma membrane receptors with TNF-α could activate an NF-KB signaling pro-inflammatory pathway that shifts macrophages into pro-inflammatory states. Previous studies have also implicated the activation of the nuclear factor kappa B (NF-KB) pathway by TNF-α in macrophages. These studies suggest that NF-KB may be one of the possible signaling pathways driving MEV-mediated M2 to M1 repolarization. Nuclear factor-kB (NF-KB) is one of the main transcription factors of M1 macrophages and regulates the expression of genes that control factors such as inflammation. Activation of NF-KB is characterized by the nuclear translocation of the p65 component of the NF-KB complex.

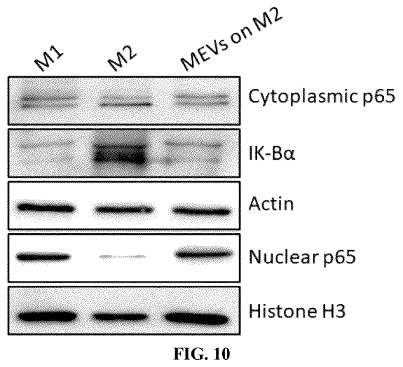

To determine if MEV delivery to M2 BMDMs activated the NF-KB signaling pathway, M2 Macrophages were incubated with MEVs for 6 h, then the cells were fractionated into cytoplasmic and nuclear fractions to study the effects of MEV delivery on the translocation of p65 subunits to the nucleus. Western blotting analysis was performed to compare the p65 content in MEV-treated M2 BMDMs using untreated M1 and M2-BMDMs as controls. Western blotting analysis indicated a significant translocation of p65 from the cytoplasm to the nucleus indicating the NF-KB pathway is activated when MEVs interact with M2 BMDMs (FIG. 10).

Improving the capability of nanovesicles to modulate macrophage polarization was sought by generating programmed nanovesicles that overexpress specific membrane-bound chemokines. Chemokines are chemotactic cytokines produced by various cells including macrophages. While chemokines are mostly known for their role in monocyte recruitment/migration, they can promote macrophage differentiation as well as polarization. For example, C—C motif chemokine ligand 5 (CCL5) has been shown to activate M1 polarization and inhibit M2 polarization. Based on the computational pathway analysis approach results in Table 2, C—C motif chemokine receptor 3 (CCR3) and CCR5 plasma membrane receptors were found to drive pro-inflammatory pathways and are among the highest-ranked pathways that polarize macrophages into pro-inflammatory states.

Therefore, MEVs were analyzed for the presence of 25 different macrophage-associated chemokines that could interact with CCR3 or CCR5 and modulate M2 macrophages toward the M1 phenotype. Only nine chemokines including C—C motif chemokine ligand 5 (CCL5), C—X—C motif chemokine ligand 9 (CXCL9), macrophage inflammatory protein 1-γ (MIP1-γ), C—C motif chemokine ligand 1 (CCL1), macrophage inflammatory protein-2 (MIP-2), C—C motif chemokine ligand 27 (CCL27), C-X-C motif-chemokine ligand 16 (CXCL16), C—C motif chemokine ligand 2 (CCL2), and monocyte chemotactic protein 5 (MCP-5) were found to be present on MEVs (FIG. 7A). The level of these observed chemokines for MEVs was compared to M2 macrophages. Several M1-polarizing chemokines including CCL2, CCL5, and CXCL9 were present at significantly higher levels in MEVs compared to M2 macrophages (FIG. 7A-7B).

The CCR3 pathway was evaluated next. CCR3 is activated by several ligands, including CCL5. Treatment of peritoneal macrophages and bone-derived macrophages with CCL5 has been shown to activate MAPK and NF-KB, which are associated with the M1 proinflammatory state. Namely, CCL5 was shown to produce proinflammatory cytokines such as TNF-α via the NF-KB pathway through the phosphatidylinositol 3-kinase (PI3K)/protein kinase B (AKT) pathway. However, CCL5 has also been reported to have a higher affinity to CCR5 than CCR3. Overexpressing CCL5 can therefore potentially preferentially activate CCR5, which is associated with polarization to an anti-inflammatory state. This may occur via the MEK/STAT3 pathway.

To reconcile these disparate outcomes, it was proposed that over-expression of CCL5 would acutely activate the CCR3 pathway and thereby drive pro-inflammatory responses in MEV-treated TAMs. To test this concept, programmed CCL-5 overexpressing HEK cell-derived nanovesicles (P(CCL5)-HNVs) were generated and the macrophage repolarization efficacy of (P(CCL5)-HNVs) was compared with unmodified HEK cell-derived nanovesicles. HEK nanovesicles themselves do not polarize M2 macrophages. Therefore, by overexpressing CCL5 on HEK nanovesicles, the role of CCL5 present in macrophage modulation can be determined.

CCL5 expression in programmed HEK cells as well as programmed nanovesicles was confirmed by western blotting (FIG. 7C). While HEK cells did not express mouse CCL5 prior to transfection, programmed HEK cells and programmed nanovesicles showed a clear band for CCL5, confirming CCL5 expression in both programmed cells and programmed nanovesicles.

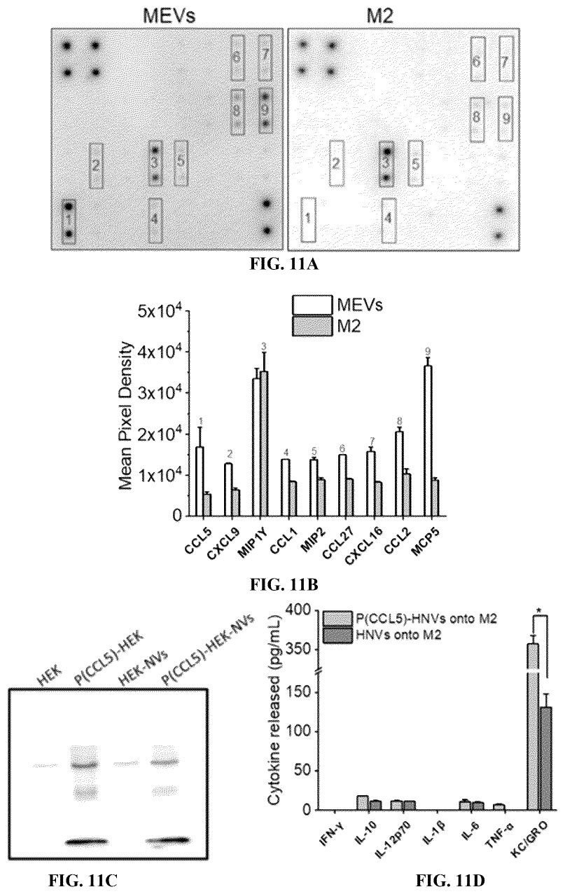

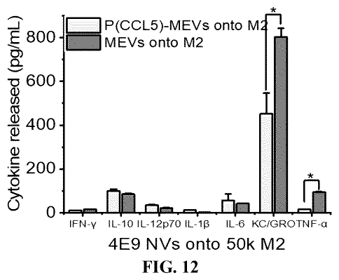

In order to compare the repolarization efficacy of programmed vesicles relative to regular HEK vesicles, 4×10⁹ vesicles were added to 50 000 M2 macrophages in culture, incubated for 24 h, and the cell culture supernatants was tested for pro-inflammatory cytokines. M2 macrophages that were left to incubate with CCL5-programmed HEK cell-derived nanovesicles (P (CCL5)-HNVs) produced roughly three times more KC/GRO compared to M2 macrophages that were incubated with regular HEK cell-derived nanovesicles (FIG. 11D). However, significant differences in the production of other cytokines by M2 macrophages was not observed. Comparable results were obtained when M2 BMDMs were incubated with CCL5-programmed nanovesicles generated using M1 macrophages (FIG. 12). These results indicate that, under similar in vitro conditions, CCL-5 programmed nanovesicles can cause higher keratinocyte chemoattractant (KC)/human growth regulated oncogene (GRO) production by M2 macrophages. However, CCL5-programmed nanovesicles do not increase other pro-inflammatory cytokine production by M2 BMDMs.

MEVs express various exosomal marker proteins, including CD54 (ICAM-1), CD63, MHCII, CD11b, and CD81 (FIG. 2C). Recent studies have shown that ICAM-1 prevents M2 polarization and inhibits tumor metastasis. ICAM1 is a cell surface glycoprotein produced in breast cancer cells following their exposure to pro-inflammatory cytokines. The pathway identified suggests that ICAM1 produces TNF-α and IFN-γ via the MAPK signaling pathway after binding and activating receptors ITGAL (integrin subunit alpha L) and ITGB2. ICAM1 also supports the expression of IL8, CCL3, and CCL4 by prolonging the stability of TNF mRNA. ICAM1 was also shown to inhibit M2 polarization by suppressing PTEN, which is a negative regulator of PI3K/AKT. Accordingly, the present inventors suggested that overexpression of pro-inflammatory ligands induced by cytokines may constitute another strategy for priming M1 phenotypes in TAMs. Therefore, programmed nanovesicles were generated that overexpress CD54 to assess their efficacy in macrophage phenotype modulation.

For this, M1 macrophages were transfected with a mammalian expressing mouse ICAM-1 plasmid, then polarized them to an M1 phenotype and used those macrophages to generate programmed macrophage-engineered vesicles (P(CD54)-MEVs). Western blotting analysis was performed to obtain the relative expression level of ICAM1 both in programmed macrophages as well as programmed MEVs relative to control. Western blotting analysis demonstrates that programmed cells and programmed nanovesicles express about tenfold higher concentrations of ICAM1 relative to their respective controls (FIG. 13A, 13B).

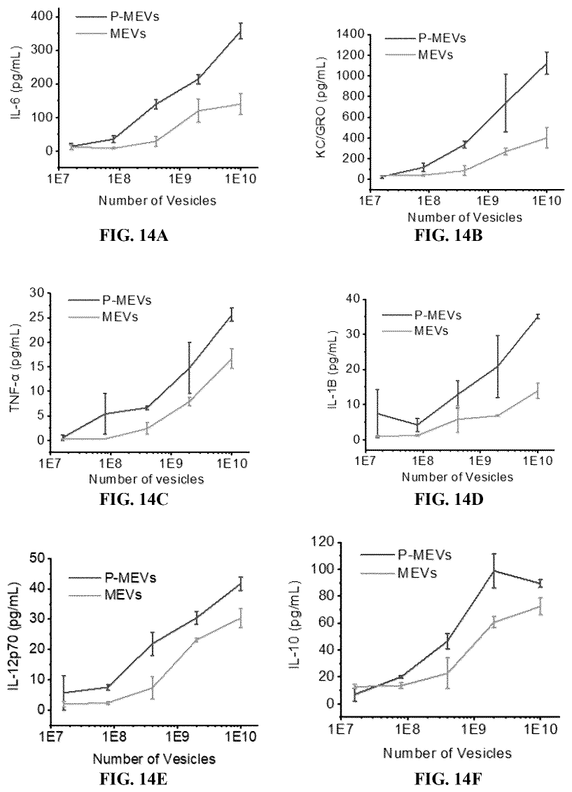

Next, the macrophage repolarization efficacy of (P(CD54)-MEVs) was compared with regular MEVs by delivering an increasing concentration of each type of MEV onto M2 macrophages and quantifying the cytokine released by MEV-treated M2 BMDMS. M2 BMDMs incubated with programmed MEVs (P(CD54)-MEVs) produced higher levels of pro-inflammatory cytokines compared to M2 BMDMs incubated with the same number of unmodified MEVs (FIG. 14). When the cytokine release by M2 BMDMs incubated with the specific concentration of MEVs (4×10⁹) was analyzed, M2 macrophages incubated with programmed MEVs produced roughly three times more KC/GRO, two times more IL-6 and IL-1β, and 25% more IL-12p70 and TNF-α compared to M2 macrophages that were incubated with non-programmed MEVs (FIG. 13C).

As a control study, CD54-overexpressing programmed nanovesicles were generated using CD54-transfected HEK cells and then delivered these nanovesicles to M2 BMDMs in culture. Similar results were obtained when M2 BMDMs was incubated with vesicles derived from HEK cells (FIG. 13D). The computational pathways analysis approach predicted that the ICAM1-mediated pathway could upregulate the expression of IL-1β, Il-6, and TNF-α in MEV-treated M2 macrophages. The experimental results indicate consistent increases in most pro-inflammatory cytokines as predicted by the computational strategy. These results further support the idea that endogenous proteins can be overexpressed in programmed nanovesicles to yield greater immunomodulatory properties compared to unmodified cell-derived vesicles.

### Example 5: Programming MEVs With Exogenous Ligands for Enhanced Macrophage Modulation

The incorporation of specific non-endogenous ligands into MEVs could yield improved levels of M2 macrophage reprogramming capability as these ligands have the potential to directly interact with receptors present on M2 macrophages and initiate downstream signaling cascades. Here, pathways were identified that are associated with TLR1 and TLR2 activation, which are toll-like receptors that dimerize upon the binding of lipopeptides. Activation of these toll-like receptors in turn primes pro-inflammatory pathways mediated by myeloid differentiation primary response 88 (MyD88).

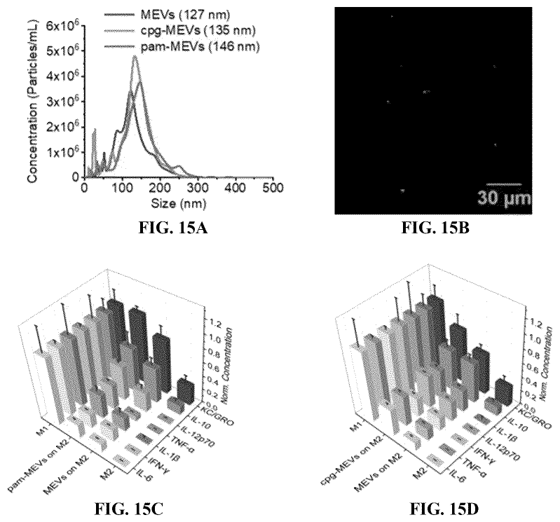

The TLR1/2/4-NF-KB signaling pathway, using a pathway analysis approach, shows the production of pro-inflammatory cytokines TNF-α, IL1B, IL6, and IL12 in two ways: PI3K-AKT signaling pathway (RAC1-PI3K-AKT-NF-KB) and mitogen-activated protein kinases (MAPK) signaling pathway. However, it also shows the production of pro-inflammatory cytokines via NF-kappa-B inhibitor alpha. TLR1/2 stimulation was therefore proposed, via compounds like N-Palmitoyl-S-[2,3-bis(palmitoyloxy)-(2RS)propyl]-[R]-cysteinyl-[S]-seryl-[S]-lysyl-[S]-lysyl-[S]-lysyl-[S]-lysine (Pam3CSK4), to determine the capacity of these receptors' activation in priming M1 polarization. To validate the strategy, macrophage-engineered vesicles were first generated from mouse pro-inflammatory M1 macrophages (MEVs) and then Pam3CSK4, a toll-like receptor 2(TLR2)/TLR1 ligand, was grafted onto the lipid bilayer membrane of the vesicles by sonication. Rhodamine-labeled Pam3CSK4 was used to confirm the successful decoration of Pam3CSK4 on the surface of vesicles. Green punctate regions in FIG. 15B, a widefield fluorescence image of MEVs that had been programmed with Rhodamine-labeled Pam3CSK4, showed the successful decoration of Pam3CSK4 on the surface of MEVs.

Nanoparticle tracking analysis was used to compare the size of these vesicles and found that Pam3CSK4 grafted vesicles (pam-MEVs) were slightly larger (146 nm) as compared to MEVs (127 nm) (FIG. 15A). The zeta potential of pam-MEVs was −16.8 mV, indicating improved stability in aqueous solution. To compare the capability of MEVs and pam-MEVs to reprogram M2 macrophages, M2 BMDMs were incubated separately with an equal number of MEVs or pam-MEVs and compared their cytokine production. While M2 macrophages show no detectable proinflammatory cytokines, those incubated with pam-MEVs showed substantial levels across most cytokines. Comparing cytokine levels typically observed for M1 polarized macrophages, pam-MEV treatment shifted M2 macrophages to 20±2% (IFN-γ), 64±20% (IL-10), 50±4% (IL-12p70), 35±7% (IL-1β), 20±2% (IL-6), 100±7% (KC/GRO) and 36±3% (TNF-α) of the average concentration seen for M1 macrophages (FIG. 15C). Similarly, M2 macrophages incubated with standard MEVs, exhibited values of 17±1% (IFN-γ), 51±11% (IL-10), 28±6% (IL-12p70), 20±8% (IL-1β), 12±1% (IL-6), 79±16% (KC/GRO) and 7±1% (TNF-α) of the average concentration seen for M1 macrophages (FIG. 15C). pam-MEVs treated M2 macrophages were more efficient at reprogramming M2 macrophages toward a proinflammatory phenotype than unmodified MEVs.

Programmed nanovesicles were also generated by incorporating small molecule agonists on the vesicle surface. Cytosine-phosphorothioate-guanine oligodeoxynucleotides (CpG-ODN), a synthetic oligonucleotide, has sequence patterns that resemble bacterial DNA and has been found to activate APCs such as macrophages and dendritic cells. CpG-ODN is recognized by the Toll-like receptor 9 (TLR-9) present in APCs. CpG-ODN interaction with TLR-9 initiates signaling pathways that causes the APC to secrete several pro-inflammatory cytokines including IFN-γ, IL-12p70, and TNF-α. Recent studies have shown that stimulation of macrophages with ODN1826, a Class B CpG oligonucleotide, increases their phagocytotic and anti-tumor activity. CpG-ODN-treated macrophages produced high levels of pro-inflammatory cytokines compared to other immune-stimulant agonists upon interaction with macrophages. While CpG-ODN is a well-known immunostimulatory agonist, its efficacy is limited by its rapid degradation and inability to effectively be delivered to the intracellular compartment of APCs.

ODN1826 was incorporated into the membrane bilayer of MEVs. To compare the capability of MEVs and cpg-MEVs to reprogram already polarized macrophages, M2 BMDMs were separately incubated with an equal number of MEVs or cpg-MEVs and cytokine production was compaired. Cpg-MEVs induced the production of cytokines to levels of 26±3% (IFN-γ), 58±11% (IL-10), 46±1% (IL-12p70), 41±3% (IL-1β), 38 +3% (IL-6), 75±18% (KC/GRO), and 43±2% (TNF-α) of the average concentration seen for M1 macrophages (FIG. 15D). This was an improvement over the polarization induced by unmodified MEVS of 7%, 1%, 18%, 10%, 26%, 18%, and 26% higher efficiency in the production of IFN-γ, IL-10, IL-12p70, IL-1β, IL-6, KC/GRO and TNF-α, respectively. These results suggest MEV efficacy can be enhanced by incorporating polarization-inducing ligands into the surface of the vesicle.

### Example 6: Generating Exosome-Mimicking Vesicles

Exosome-mimicking vesicles can be generated from BMDMs with high yield using nitrogen cavitation. Interactions of endogenous ligands present on the membrane bilayer of a vesicle with their corresponding receptors present on the target macrophage cause anti-inflammatory macrophages to repolarize toward a pro-inflammatory phenotype. Cell-engineered nanovesicles can be programmed to overexpress specific ligands on their surface that improve targeting and repolarization efficacy. Programmed nanovesicles, when interacting with the M2 macrophages can elicit enhanced immunomodulatory properties compared to non-programmed vesicles. Programmed vesicles exhibited negative zeta potentials (Table 3) indicating long-term stability in solution. This shows that programmed M1 macrophage-engineered nanovesicles have the potential to be used as a therapeutic platform to achieve enhanced re-polarization of tumor-supportive M2 macrophages toward a tumor-killing M1 phenotype.

TABLE 3
Zeta potential values measured for various programmed vesicles.
Negative zeta potential values of these programmed vesicles
indicate improved stability in aqueous solution.

| Serial No. | Macrophage-Engineered Vesicles | Zeta Potential (mV) |
|---|---|---|
| 1 | MEVs | −5.2 |
| 2 | P(TNF)-MEVs | −9.2 |
| 3 | P(CD54)-MEVs | −5.5 |
| 4 | pam-MEVs | −16.8 |
| 5 | cpg-MEVs | −9.3 |

Macrophage polarization exists in a continuum ranging across a spectrum from proinflammatory to anti-inflammatory and is most often characterized by cytokine release. While several strategies have been demonstrated herein for enhancing the ability of MEVs to repolarize macrophages toward a proinflammatory phenotype, the extent and the profile of the proinflammatory properties (e.g., cytokine release) vary across the different ligands and proteins featured on the surface of the vesicle. The specific cytokines increased by the ligands are likely reflective of the signaling pathways initiated through interactions with the target macrophage.

The ideal strategy for programming MEVs for their ability alter macrophage phenotype may require a mixture of surface functional groups to enhance the overall proinflammatory properties or may require a tailored approach that utilizes ligands that specifically elicit the production of one or more cytokines. The studies described herein establish that surface proteins on MEVs are responsible for their inherent immunomodulatory properties. By introducing specific characteristics on the surface of the vesicle's membrane, the process of repolarization can be adjusted and directed toward either a pro-inflammatory or anti-inflammatory state.

The capability to shift macrophages between polarization phenotypes offers a promising method that can be applied in treatments for several diseases. For instance, redirecting tumor-associated macrophages to a pro-inflammatory state holds promise as a strategy to increase the effectiveness of immunotherapy in treating cancer. Similarly, transforming pro-inflammatory macrophages into anti-inflammatory phenotypes could be a favorable approach to mitigate the potential neurotoxic effects associated with M1 macrophages, offering potential treatments for a wide range of diseases related to inflammation as well as conditions such as spinal cord injury.

Additionally, the technique for generating vesicles and subsequently functionalizing their surface features is readily scalable to large-scale production. The studies here utilized a relatively small volume of cells (10 mL for ˜100 million cells). Similar nitrogen cavitation vessels with 10 to 100 times the capacity are readily available. Thus, the approach is readily scalable to production levels.

### Example 7: Animals

Two to five-month-old C57/BL6 mice were used to harvest bone marrow cells. Mice that were initially purchased from the Jackson Laboratory and were appropriately housed in IVC with a sufficient supply of food and water. All experiments were conducted in accordance with National Institutes of Health guidelines that were authorized by the University of Kentucky's Institutional Animal Care and Use Committee (IACUC: Kolesar 2017-2674).

### Example 8: Cell Culture

Mouse bone marrow-derived macrophages (BMDMs) were harvested from two to five-month-old mice following previously published methods. Bone marrow from the femurs and tibias of mice was first flushed and then homogenized using Dulbecco's Modified Eagle's Medium (DMEM) media. The solution of cells was then centrifuged at 1000×g for 5 min to collect the cell pellet. Red blood cell (RBC)-lysis buffer containing 0.15 M NH4Cl, 10 mM KHCO3, and 0.1 mM Na4EDTA was then used to lyse the erythrocytes present in the cell pellet. Erythrocyte-free monocytes were then cultured in a 75-cm2 culture flask containing 12 mL of cell culture medium. Mouse bone marrow-derived macrophage cell culture medium contained RPMI media supplemented with 20% cell-culture supernatant obtained from sL929 cells, 10% fetal bovine serum (FBS), 5% penicillin/streptomycin, 1% glutamine, 1% HEPES, and 0.001% 2-mercaptoethanol. The cell culture medium was changed every two days. On day 7, cells were re-seeded at a cell density of 1×106 cells mL-1 in Dulbecco's modified eagle medium (DMEM) media supplemented with 10% FBS, 1% glutamine, and 1% penicillin/streptomycin and stimulated to M1-macrophages using LPS (20 ng mL-1) and IFN-γ (20 ng mL-1) or to M2-macrophages using IL-4 (20 ng mL-1).

### Example 9: Vesicles Isolation

Previously published methods (Yurkin et al. (2017), Snell et al. (2019), Choo et al. (2022), Neupane et al. (2021)) were followed to generate pro-inflammatory MEVs. Briefly, to generate MEVs, used fully differentiated pro-inflammatory (M1) macrophages were used. First, cells were rinsed with PBS and then resuspended in PBS containing one protease inhibitor tablet per 10 ml of 1×PBS buffer. The cell slurry was then transferred to a nitrogen cavitation vessel on ice and subjected to 300 psi of nitrogen gas pressure for 5 min. The rapid release of pressure caused the fragmentation of cells and the subsequent formation of vesicles. The nitrogen cavitation-obtained cell lysate was centrifuged at 4000×g for 10 min at 4° C. The pellet obtained after centrifugation was discarded, and the supernatant was centrifuged again for 20 min at 10 000×g and 4° C. The obtained supernatant was ultracentrifuged at 100 000×g for 60min at 4° C. to obtain a pellet containing MEVs. The pellet was then resuspended in phosphate buffered saline (PBS) to release MEVs in the solution.

### Example 10: Nanoparticle Tracking Analysis

Nanoparticle tracking analysis (NTA) was used to determine the size and concentration of MEVs in samples using a NanoSight NS300 equipped with NTA analytical software and a 488 nm laser. Samples of MEVs were diluted one thousand times with ultrapure water, and five 30-second videos were recorded for analysis. The parameters for the analysis software remained constant for all measurements of any given sample.

### Example 11: Transmission Electron Microscopy Imaging of MEVs

Vesicles were resuspended in PBS and fixed in 2% glutaraldehyde. Copper TEM grids with an ultra-thin carbon layer and lacey film (Ted Pella, Inc) were glow discharged at 10 mA for 15 s. Vesicles were deposited on the grids for 1 min. The excess solution was removed, and vesicles were negatively stained with 2% uranyl acetate for 20 s. Excess uranyl acetate was removed, and grids were air-dried. Vesicle micrographs were collected using a Talos F200X TEM (Thermo Scientific) operating at 200 kV accelerating voltage. The contrast was enhanced by inserting a 50 μm objective aperture.

### Example 12: Western Blotting

Vesicle pellets and cells were initially treated in the radioimmunoprecipitation assay (RIPA) buffer (150 mM NaCl; 1% Triton X-100; 0.5% sodium deoxycholate; 0.1% SDS; 50 mM Tris pH 8.0; 1× protease inhibitor cocktail (Roche)). Each protein sample (50 μg) was resolved on 12% polyacrylamide gels and then transferred to a nitrocellulose membrane. For comparative experiments, an equal protein from various samples was loaded per lane. The membrane was then blocked for 1 hour with 5% milk in TBST and incubated with primary antibodies at room temperature for 2 h with constant shaking. After washing and removal of the primary antibody, the HRP-conjugated secondary antibody was added for 1 h. The membranes were washed with TBST and the protein bands were developed using chemiluminescent detection.

### Example 13: Cytokine Quantification

Macrophages were seeded at a density of 50 000 in 96 well plates and then stimulated to M1 or M2 phenotypes as discussed in the previous section. For the cytokine analysis, macrophage conditioned medium (MCM) was collected after 24 hours of stimulation or treatment with MEVs. The MCM was frozen at −80° C. until analysis. Following the manufacturer's instructions from Meso Scale Discovery (MSD), a mouse seven-plex pro-inflammatory cytokine test was conducted to simultaneously detect IFN-γ, IL-10, IL-12p70, IL-1β, IL-6, KC/GRO, and TNF-α present in the MCM. Calibrators (50 μL) and MCM were used for cytokine analysis. MESO SECTOR imager from Meso Scale Delivery was used to analyze the MSD plate for pro-inflammatory cytokines. All samples were run in triplicates and used vesicle suspensions in PBS to measure the proinflammatory cytokines present in the vesicle suspension. In some experiments, the same protocol as previously discussed was used to measure the concentration of TNF-α in the MCM using a single spot well plate.

### Example 14: Chemokine Quantification

Chemokines present on the MEVs were detected using Mouse Chemokine Array C1 (Ray Biotech, Code AAM-CHE-1-2) and semi-quantified following the manufacturer's protocol. Briefly, chemokines were first extracted from MEVs and M2 macrophages, a control, using cell lysis buffer as provided by the manufacturer. The protein concentration in MIEVs and M2 samples was measured using a UV/VIS spectrophotometer. For proteomic analysis, first, the antibody arrays were incubated in 2 mL of blocking buffer for half an hour with constant shaking at room temperature. After half an hour, the blocking buffer was aspirated off and ≈500 μg of the protein samples were loaded into each well of an incubation array and left to incubate for 3 h at room temperature. Following several washes, a biotinylated antibody cocktail was added to each well and incubated for 2 hours at room temperature. The biotinylated antibody cocktail was then aspirated off, followed by multiple washings. After this, 1× horseradish peroxidase (HRP)-Streptavidin was added to each well and incubated for 2 h at room temperature. After multiple washings, 500 μL of detection buffer mixture was added to each membrane and visualized by chemiluminescent detection. The immunoblot images were analyzed using ImageJ software.

### Example 15: Coding Information

The code for this study is provided as a bitbucket repository that is currently available at bitbucket.org:pkhlab/pathwayanalysis.git. A website for its usage and an example ipython notebook is provided at bending456.github.io/Macrophage/and colab.research.google.com/drive/12jtuAzQAvVtMoYm2EhInrxKgyit99Vc9?usp=sharing.

### Example 16: mRNA Analyses

To find genes that were highly expressed or repressed in M1 macrophages, the authors searched for mRNA data that was publicly available on the NCBI GEO website. The mRNA data was retrieved from the gene expression microarray experiment in NCBI with GEO accession number GSE57614, PMID: 25 799 240 The study included Transcriptomic analysis of human polarized macrophages, under four conditions: Resting macrophage (M0), M1 (stimulated using IFN-γ, and LPS), M2a (by IL-4) and M2c (by IL-10). Transcription profiles were taken after 3-time points: 6, 12, and 24 h, each with 3 replicates. M2a samples induced by IL-4 were chosen over M2c induced by IL-10. Macrophages were also stimulated for 24 h, so the use of the 24-hour dataset instead of the 6- and 12 h was chosen to stay as close to the experiment as possible. The online statistical tool GEO2R was used to examine the raw gene expression data, which utilized R/Bioconductor and Limma package v3.26.8. Inbuilt statistical methods such as the t-test and Benjamini and Hochberg (false discovery rate) were used to determine the DEGs. Two comparisons were made: 1) M0 versus M1 and 2) M2 versus M1. From these two lists of DEGs, genes with an adjusted p-value less than 0.05 were considered significant. The two lists were then processed to find common genes found in both lists. This list was used to cross-reference proteins localized in the plasma membrane.

### Example 17: Plasma Membrane Localization

To find proteins localized to the plasma membrane, the authors turned to UniProt to look for annotations of proteins in the plasma membrane. Specifically, the authors searched for a location as “cell membrane” and function as “receptor”. Annotations for plasma membrane proteins were downloaded, filtered, and processed.

### Example 18: Network Analysis

A graph-theoretic approach was adopted to evaluate macrophage signaling pathways. The signaling networks comprised nodes representing intracellular or plasma membrane proteins, while edges linking those proteins symbolized protein-protein interactions or communication via second messengers. The advantage of this representation is that there were well-established algorithms for identifying optimal pathways that link two arbitrary nodes, such as a path that links receptor activation to a cytokine response typical of M1 macrophages. The key steps to the approach were 1) the duration of signaling networks from databases and 2) pathway searching via network analysis algorithms. For 1), candidate pathways implicated in macrophages from databases that include the Kyoto Encyclopedia of Genes and Genomes (KEGG) and Wikipathways were manually collected. Cytoscape was used to merge and curate pathways into a broad network of protein-protein interactions. The resulting network topology was exported for analysis using NetworkX. NetworkX was a comprehensive Python library for performing routine graph analyses, including the identification of ‘minimum first paths’ that linked a given receptor to an M1-associated cytokine gene product. RNAseq and immunohistochemistry data reported in the literature or by collaborators to align the curated network topologies with properties specific to a given macrophage subtype were additionally incorporated. Altogether, 1) and 2) yielded sets of pathways and the receptors that activated them in a rank-ordered list.

### Example 19: STRING Network Extension

Using the STRING database extension software, edges were added to refine the network. This resulted in the pathways used for the search protocol. To infer whether an edge was agonistic versus antagonistic potential, it was assumed that if two genes had positively correlated changes in mRNA expression, the edge was agonistic; otherwise, a negative correlation was assumed to be antagonistic. However, most were deemed insignificant.

### Example 20: Ligand Incorporation into MEVs

MEVs were generated from 150 million M1 macrophages and resuspended in a 500 μL solution containing ligands (Pam3CSK4, rhodamine-labeled Pam3CSK4, or CpG-ODN) at 1 mg mL-1. The ligand-MEV solution was then sonicated using a Q125 sonicator with a 0.125″ tip with the following settings: 20% amplitude, 20 cycles of 30 s on/off for 10 min. The MEV-ligand solution was allowed to cool down on the ice for two minutes between each cycle. After completion of the sonication cycle, the ligand-MEV solution was left to incubate on ice for 60 min to allow the recovery of the MEV membrane. The ligand-decorated MEVs containing solution was diluted to 4 mL in PBS and subjected to ultracentrifugation at 100 000×g for 60 min at 4° C. to collect the pellet containing ligand-decorated MEVs, which was washed with 1 mL of PBS twice and resuspended in 500 μL of regular macrophage media. The number of MEVs was determined using NTA. 1×1011 MEVs were then added into each well of a 96-well plate containing 50 000 M2 macrophages in 100 μL of replating media. The plate was left to incubate for 24 h at 37° C. After 24 h, MCMs were collected and used for pro-inflammatory cytokine analysis.

### Example 21: RelA Translocation Assay

The activation of the NF-KB pathway by MEV was measured by comparing the quantity of the p65 subunit (RelA) that was translocated into the nucleus using a nuclear translocation assay. The NF-KB Assay Kit (FIVE photon Biochemicals, San Diego, CA) was used to fractionate M1 macrophages, M2 macrophages, and M2 macrophages treated with MEV into nuclear and cytoplasmic fractions. The amount of p65 in both the nuclear and cytoplasmic fractions was determined by western blotting.

### Example 22: Confocal Imaging

A Nikon AIR confocal microscope was used for confocal imaging of the cells that was programmed to overexpress specific ligands on their surface. Images were analyzed using Nikon Elements image processing software.

### Example 23: Transfection

HEK cells were transfected using Lipofectamine 2000 reagent (Invitrogen) using the manufacturer's protocol. The authors used 2-5-month-old wild-type C57BL/6 mice to isolate bone marrow monocytes. Monocytes were then differentiated into macrophages. On day five, macrophages were transfected with a plasmid. Macrophages were transfected using the jet PEI-Macrophage in vitro DNA transfection reagent following the manufacturers protocol. Transfection efficiency was compared by confocal imaging and western blotting.

### Example 24: Statistical Analysis

Statistical analyses were carried out using Origin. Data were reported as the mean±standard deviation of the mean (SEM). At least three separate experiments were conducted for each condition (n=3). A one-way ANOVA was done to determine statistical significance with Tukey's post hoc analysis. The results were considered statistically significant if the p-value was less than or equal to 0.01.

### Example 25: erMEVs, pmMEVs Characterization

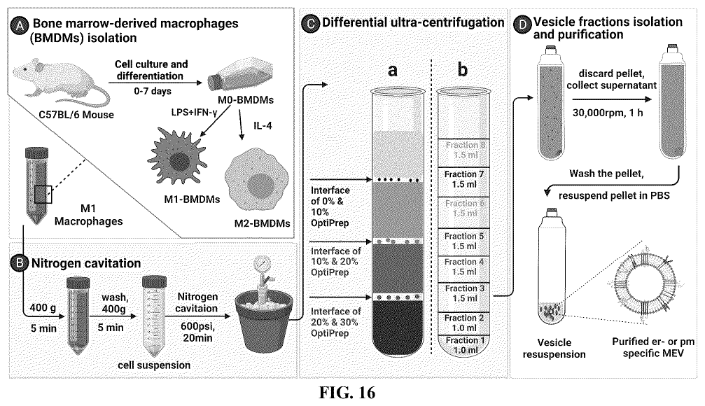

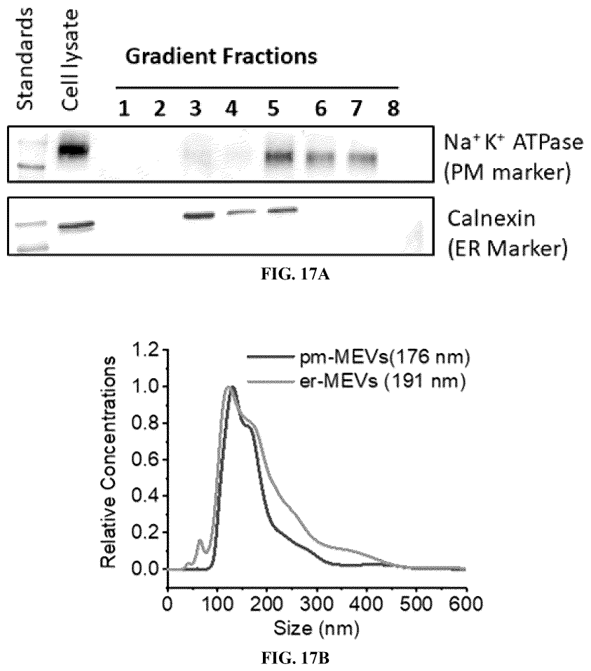

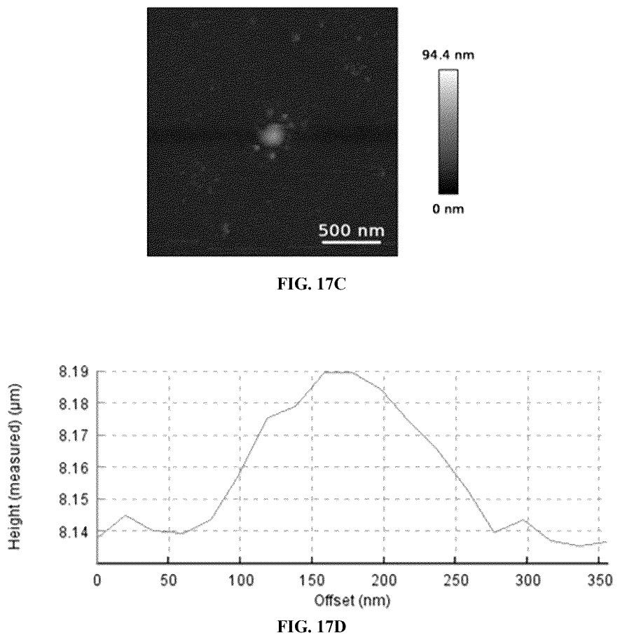

Endoplasmic reticulum was generated and separated from plasma membrane specific pro-inflammatory macrophage engineered vesicles by employing nitrogen cavitation and ultracentrifugation through a gradient as in FIG. 16 and as discussed in Moonschi, et al. (2018). Nitrogen cavitation is a technique that has been widely used to mechanically disrupt cell-associated membranes into nano-sized fragments. See, e.g., Harvey, et al. (2022), Dong, et al. (2019), and Gao, et al. (2022). While vesicles are generated from practically every subcellular organelle, differences in membrane densities due to dissimilar phospholipid and protein content allow them to be separated based on organelle of origin. Moonschi, et al. (2018). For example, endoplasmic reticulum generated vesicles exhibit greater densities compared to vesicles generated from the plasma membrane. See, van Meer, et al. (2008). Western blotting analysis was performed to confirm the presence of endoplasmic reticulum or plasma membrane specific protein markers for each set of vesicles isolated (FIG. 17A). Anti-sodium potassium ATPase (Atpla) primary antibody was used for the plasma membrane marker and anti-calnexin (Canx) primary antibody was used for the endoplasmic reticulum marker. Western blotting results indicated that the plasma membrane marker was present in gradient fractions from 5 to 7, and the endoplasmic reticulum marker protein was found to be present in the fractions from 3 to 5 (FIG. 17A). Therefore, fractions 3 and 4 primarily contain endoplasmic membrane derived vesicles while fractions 6 and 7 primarily contain plasma membrane derived vesicles.

For the experiments, fraction 3 was selected for endoplasmic reticulum derived vesicles and fractions 6 and 7 for plasma membrane derived vesicles. Nanoparticle tracking analysis (NTA) was performed using NS-300 to determine the vesicle yield and the size distribution of each type of MEVs. Using multiple particle tracking analysis, it was determined that 45 million pro-inflammatory M1 macrophages yields roughly 6×10¹⁰ erMEVs and 2×10¹¹ pmMEVs. In addition, the size distribution of erMEVs (mean diameter=191.1 nm) was found to be slightly greater than the size distribution of pmMEVs (mean diamete =176.3 nm) (FIG. 17B). While erMEVs were slightly larger than pmMEVs, the size range for both kinds of vesicles still falls in the range of exosomes. See Wang, et al. (2021).

To further characterize the vesicles, their surface charge was measured when suspended in PBS buffer. erMEVs and pmMEV exhibited a surface charge (zeta potential) of −11.6±1.8 mV and −7.4±2.8 mV, respectively. The zeta potential determined for the erMEVs and pmMEVs falls in the range of −5 mV to −20 mV which is similar to the zeta potential that has been reported for endogenously released extracellular vesicles (EEVs), including exosomes. See Jang, et al. (2022). A negative zeta potential like that exhibited by erMEVs and pmMEVs indicates that they should be stable in the aqueous solution. Vesicle size was further confirmed using atomic force microscopy (AFM), where the diameter of individual vesicles was found to be approximately 150 nm (FIG. 17C, 17D).

These studies indicate that pro-inflammatory macrophage derived subcellular specific vesicles can be generated using nitrogen cavitation with a similar size to EEVs.

### Example 26: In Vitro Delivery of erMEVs and pmMEVs

Previous studies have shown that endogenously released extracellular vesicles exhibit preferential targeting specificity to the cell of origin. For example, exosomes released from cancer cells are preferentially taken up by the cancer cells where they originated. See, e.g., Kim, et al. (2019). Similar targeting specificity has been seen for cell derived vesicles. Jang, et al. (2013); Neupane, et al. (2021). Studies were conducted to determine if there were differences in the targeting of vesicles derived from the er and the pm.

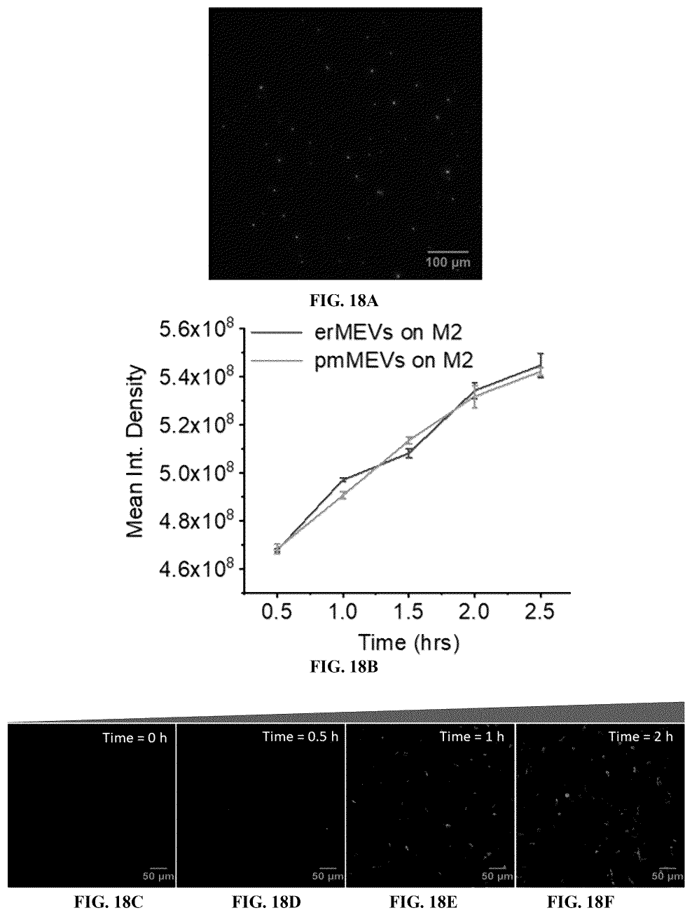

Endoplasmic reticulum (er) and plasma membrane (pm) specific MEVs were generated from M1 (IFN-γ+LPS) BMDMs and labeled with a lipophilic dye, DiI. Upon incubation of DiI with vesicles, the fluorophore embeds in the bilayer rendering them fluorescent. Fluorescently labeled vesicles were then purified from the free dye using a size exclusion column. The fluorescence image (FIG. 18A) showing red punctuated regions indicates the successful labeling of vesicles with DiI. Next, experiments were performed to compare the efficiency of delivery of fluorescently labeled erMEVs and pmMEVs to anti-inflammatory M2 macrophages (FIG. 18B). The fluorescence signal at various time points over 2.5 hours was used to determine the level of uptake. While M2 macrophages are initially non-fluorescent, a gradual increase in fluorescence signal can be observed after labeled-vesicle uptake (FIG. 18C-18F). Both erMEVs and pmMEVs were efficiently delivered to M2 macrophages, and there was no significant difference in the uptake of labeled erMEVs and pmMEVs by M2 macrophages. These results demonstrate that erMEVs and pmMEVs can be successfully delivered to anti-inflammatory M2 macrophages with efficient uptake of both types of vesicles but no apparent difference in efficiency.

### Example 27: erMEVs and pmMEVs Mediated Macrophage Phenotype Modulation

Previous studies have demonstrated that endogenously released exosomes from pro-inflammatory macrophages are able to re-program macrophage phenotypes from anti-inflammatory (M2) to pro-inflammatory (M1)-like phenotypes and vice versa. See Choo, et al. (2018), Cheng, et al. (2017), Kim, et al. (2019). Similar results have been seen for cell derived vesicles. Neupane, et al. (2021). Studies were conducted to determine whether there were differences in immunomodulatory activity of vesicles from different organelles. Both erMEVs and pmMEVs were generated and delivered them to M2 macrophages, and the cytokine released by M2-macrophages that had been incubated with erMEVs or pmMEVs was analyzed.

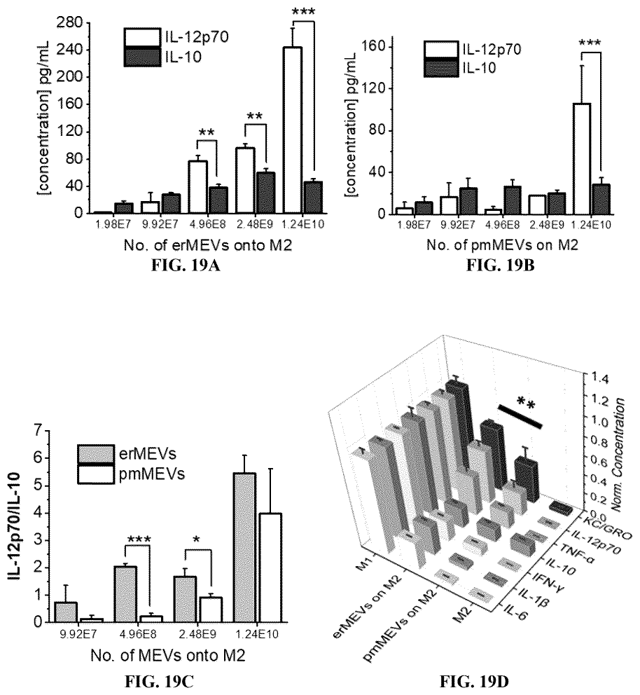

Increasing concentrations of erMEVs and pmMEVs (1.98×10⁷ to 1.24×10¹⁰) were added to 50,000 M2 BMDMs in culture and incubated them for 24 hours at 37° C. Following that, macrophage conditioned media was collected from each experimental condition and analyzed for the presence of seven different pro-inflammatory cytokines in the cell culture supernatant, including IFN-γ, IL-10, IL-12p70, IL-1β, IL-6, KC/GRO, and TNF-α. Both erMEVs and pmMEVs can re-polarize M2 macrophages towards an M1-like phenotype in a concentration-dependent manner (FIG. 19A, 19B). In addition, upon incubation of M2 macrophages with an equal concentration of erMEVs or pmMEVs, erMEVs induced significantly higher pro-inflammatory cytokine production by M2 macrophages compared to pm-MEVs (FIG. 19D).

M2 macrophages are well known for expressing high levels of the anti-inflammatory cytokine IL-10 in comparison to pro-inflammatory macrophages, which express higher levels of IL-12. Therefore, IL-10: IL-12 is often a defining feature of M1 and M2 macrophage. Zhang, et al. (2019). The ratio of IL-10 to IL-12 production has also been linked to tumor progression, with increased IL-10 to IL-12 production leading to an increase in metastatic effect. Miteva, et al. (2014); Liu, et al. (2016). Accordingly, the ratio of IL-12 to IL-10 production by MEV-treated M2 BMDMs was investigated. The results demonstrate that both erMEVs and pmMEVs induced increased production of IL-12 as compared to IL-10, thereby maintaining the ratio of IL-12 to IL-10 greater than one further indicating that erMEVs or pmMEVs-M2 macrophage interaction drives M2 macrophages towards a proinflammatory phenotype (FIG. 19C).

Furthermore, when M2 macrophages were incubated with erMEVs or pmMEVs in a dose dependent manner, it was found that at a lower dose of 9.92E7 vesicles there is no significant difference between the proinflammatory cytokines released by M2 macrophages incubated with erMEVs or pmMEVs. However, with increasing concentrations erMEVs exhibit greater repolarizing properties compared to pmMEVs. Additionally, the ability of erMEVs or pmMEVs to repolarize M2 macrophages towards an M1 like phenotype was confirmed using western blotting analysis. For this, M2 macrophages that originally do not express CD86, a M1 macrophage protein marker were left to incubate with equal number of erMEVs or pmMEVs. After 72 hours, erMEVs treated macrophages expressed CD86 to a greater extent compared to pmMEVs treated M2 macrophages. Interestingly, M2 macrophages that originally expressed CD206, a M2 macrophage marker, exhibited a significant decrease in CD206 expression after interacting with both erMEVs and pmMEVs. These results further validated that erMEVs are more potent at reprogramming M2 macrophages towards M1 like phenotype.

Nitrogen cavitation followed by density gradient separation can be used to generate organelle specific vesicles at high yields. While both ER and plasma membrane specific vesicles can execute M2 to M1 reprogramming, erMEVs are better at doing so. As noted above, a similar preparation method was used with vesicles from the entire cell without separation. Neupane et al. (2023). While the separation of vesicles by subcellular organelle is not prohibitively expensive, it is more time consuming. Membrane proteins in the cell surface are likely responsible for the ability to repolarize. These results show that M1 macrophage derived erMEVs likely have a protein composition more potent in repolarizing M2 macrophage polarization that could elicit macrophage reprogramming in the tumor microenvironment.

### Example 28: erMEVs and pmMEVs Repolarize M2 Macrophages Co-Cultured With Cancer Cells

The tumor microenvironment comprises not only the tumor cells but also various other cells, including tumor associated macrophages (TAMs). TAMs present in the tumor microenvironment are known to execute anti-tumor immune suppression as well as tumor proliferating functions. Boutilier, et al. (2021). Tumor associated macrophages have been implicated as resembling M2 macrophages Schweer, et al. (2022). The ability to re-program M2 macrophages present in the tumor microenvironment to an M1-like phenotype would therefore help to re-store the anti-tumor properties of the macrophages. The capability of er-MEVs and pm-MEVs to re-program M2 macrophages that had been co-cultured with carcinoma cells was determined. For the co-culture experiments, A549, a lung cancer cell line, and Caov3, an ovarian adenocarcinoma cell line, were used. erMEVs (1.2×10¹⁰) or equal number of pmMEVs were added to each well of a 96-well plate that had 20,000 carcinoma cells and 40,000 M2 BMDMs in culture and allowed to incubate for 48 hours. Next, the cell culture media was collected from each experimental condition and analyzed for the presence of the pro-inflammatory cytokine TNF-α in the cell culture supernatant.

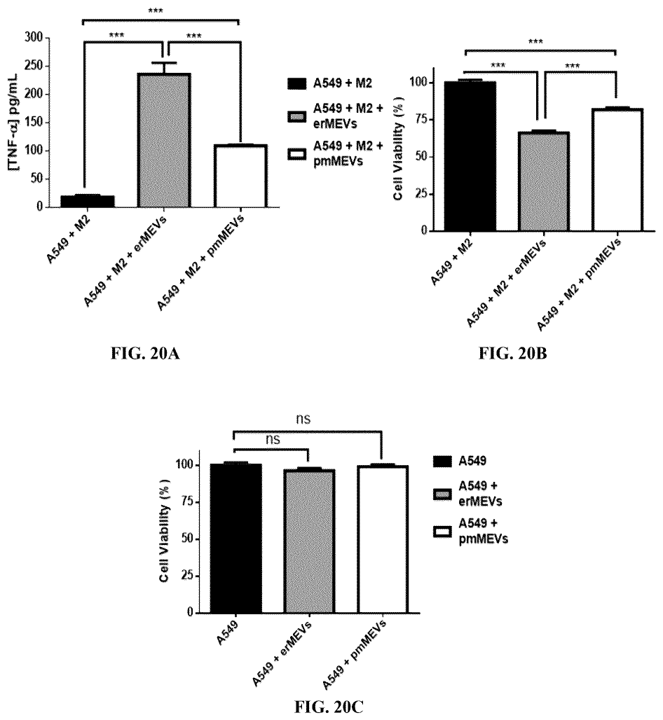

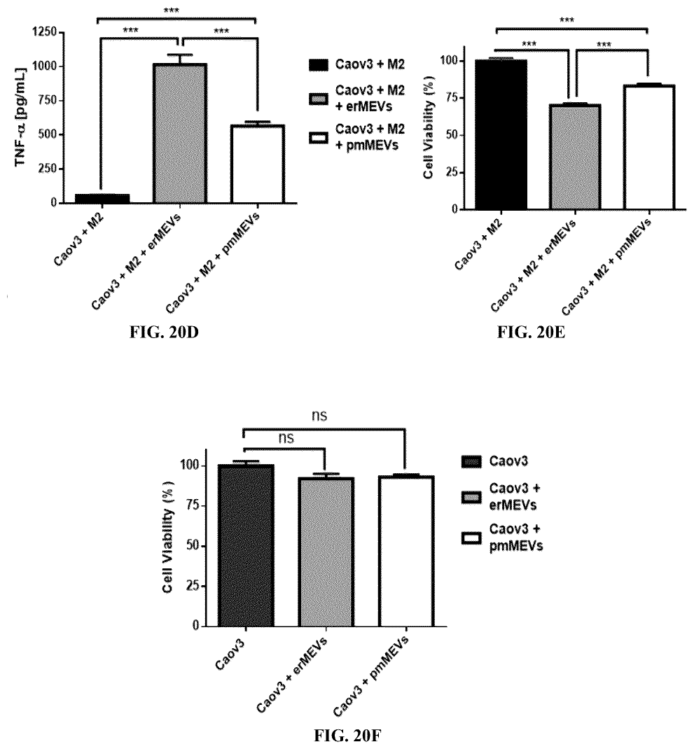

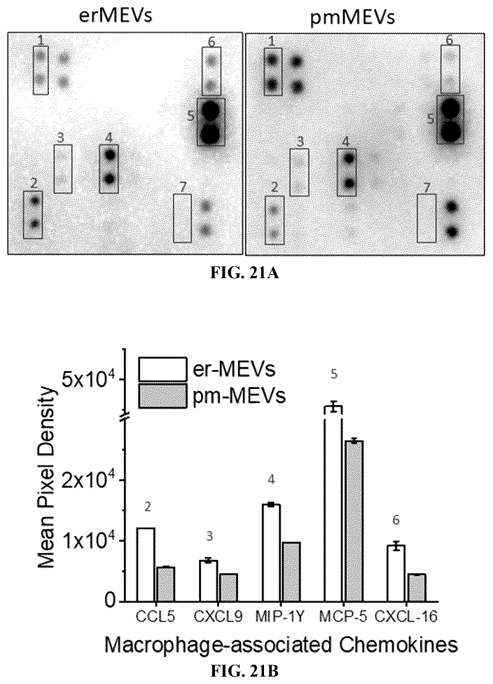

Cells co-cultured with either erMEVs and pmMEVs were found to secrete increased levels of TNF-α, demonstrating that both erMEVs and pmMEVs can still repolarize M2 macrophages that were co-cultured with cancer cells towards an M1-like phenotype. Under similar conditions, the presence of erMEVs caused M2 macrophages to produce significantly more TNF-α (p<0.001) than the presence of pmMEVs (FIG. 20A, 20D). These results are similar to what was observed when erMEVs and pmMEVs were delivered to M2 macrophages alone (FIG. 19D).

The capability of erMEVs and pmMEVs to inhibit cancer cell growth under co-culture conditions was determined. 1.2×10¹⁰ erMEVs or pmMEVs were incubated with 20,000 carcinoma cells and 40,000 M2 BMDMs in co-culture for 48 hours. Cell viability was then determined using a fluorescence assay (alamar blue) in which the fluorescence signal correlated with the number of live cells. By a 48-hour time point, erMEVs resulted in about 30% cell death, while pmMEVs resulted in about 15% cell death (FIG. 20B, 20E). These results demonstrate that, under in vitro conditions, erMEVs are more effective at inhibiting cancer cell viability than the same concentration of pmMEVs. The cell viability assay results are similar to the M2 macrophage reprogramming efficacy results of erMEVs and pmMEVs. M1 macrophages are known to execute anti-tumoral functions by releasing pro-inflammatory cytokines and oxidative metabolites such as nitric oxide. Therefore, the higher cancer cell death prompted by the presence of erMEVs in the co-culture conditions may be attributed to the presence of higher levels of proinflammatory factors released by re-programmed M1-like macrophages in the macrophage conditioned media.

### Example 29: Proteomic Analysis of erMEVs and pmMEVs

After confirming the capability of erMEVs and pmMEVs to re-program anti-inflammatory macrophage to pro-inflammatory (M1)-like phenotype, proteomic analysis studies were performed to identify the possible endogenous ligands present on the erMEVs or pmMEVs which are mediating the re-polarization. As erMEVs or pmMEVs are generated by fragmenting pro-inflammatory macrophages, erMEVs and pmMEVs contain ligands belonging to the parent pro-inflammatory (M1) macrophage. Therefore, analysis was focused on cytokines, chemokines, heat shock proteins, and other endogenous ligands associated with M1 macrophages that could be present on the vesicles. Cytokines produced by the macrophages are known to interact with their corresponding receptors present on the immune cell and drive changes in immune cell phenotype. For example, cytokines IL-12, TNF-α are known to program macrophages towards a pro-inflammatory phenotype. Dostert, et al. (2019). erMEVs and pmMEVs were analyzed for seven pro-inflammatory cytokines including IFN-γ, IL-10, IL-12p70, IL-1β, IL-6, KC/GRO and TNF-α present in the vesicle suspension using a mouse pro-inflammatory seven-plex ELISA assay from Meso Scale Discovery (MSD). In addition, vesicles that releases any cytokines entrapped inside of the vesicles in the solution were freeze ruptured. erMEVs and pmMEVs were found to contain very low concentrations of pro-inflammatory cytokines in the interior of the vesicles or in the solution. Comparing the cytokine content of erMEVs and pmMEVs, erMEVs have slightly higher concentration of TNF-α compared to pmMEVs.

M1 and M2 macrophages produce different chemokines that execute distinct functions. For example, M1 macrophages secrete the Th1 cell attracting chemokine CXCL9 and CXCL10 whereas M2 macrophages secrete chemokines including CCL17, CCL22 and CCL24. Kim et al. (2019). Recent studies have shown that macrophages can be completely repolarized from M2 to M1-phenotype due to exposure to relevant chemokines. Kim et al. (2019). Thus, the presence of chemokines in the MEVs has the potential to execute macrophage reprogramming functions.

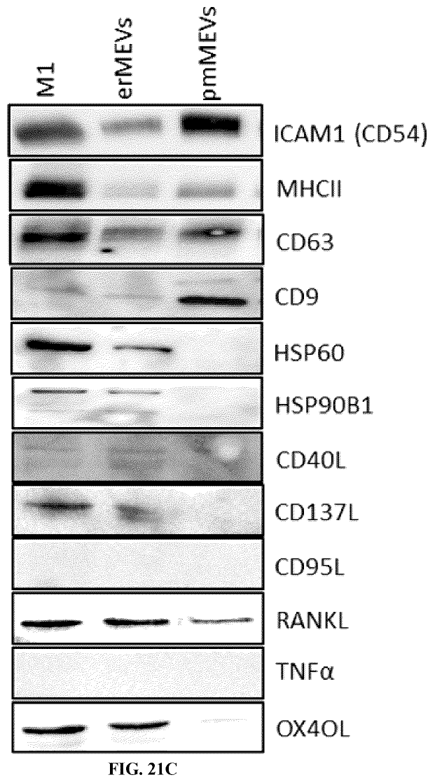

A mouse chemokine array that simultaneously measured the presence of 25 different chemokines was used. Using this assay, it was found that only five chemokines including C—C motif chemokine ligand 5 (CCL5), C—X—C motif chemokine ligand 9 (CXCL9), macrophage inflammatory protein 1-7 (MIP1-γ), C—X—C motif chemokine ligand 16 (CXCL16), and monocyte chemotactic protein 5 (MCP-5) were present on MEVs. Taking equal concentration of the protein, the level of these chemokines for er-MEVs was compared to that of pm-MEVs. Several M1-polarizing chemokines including CCL5, and CXCL9 were found to be present at significantly higher levels in erMEVs compared to pmMEVs (FIG. 21A, 21B).

Several protein ligands associated with M1 macrophages that could be present on erMEVs and pmMEVs and regulate MEV uptake and MEV-mediated macrophage repolarization were next sought to be identified. These ligands include tetraspanins, adhesion molecules, heat shock proteins, and ligands for tumor necrosis factor superfamily receptors. Both erMEVs and pmMEVs were found to express ICAM1, CD9, CD63, and MHCII molecules (FIG. 21C). However, heat shock proteins including HSP60, and HSP90B1 are present only on erMEVs (FIG. 21C). Similarly, erMEVs expressed higher levels of CD40L, CD137L, RANKL, and OX40L compared to pmMEVs (FIG. 21C). In addition, western blotting results demonstrate CD95L is absent on both erMEVs and pmMEVs. Several already known M1 polarizing endogenous ligands are present to a greater extent in erMEVs compared to pmMEVs. These results indicate that it could be the interaction of such ligands with the receptors expressed in M2 macrophages that lead to erMEVs exhibiting higher repolarizing capability compared to pmMEVs.

### Example 30: Ligand-Receptor Interactions Mediates M2 to M1 Macrophage Repolarization

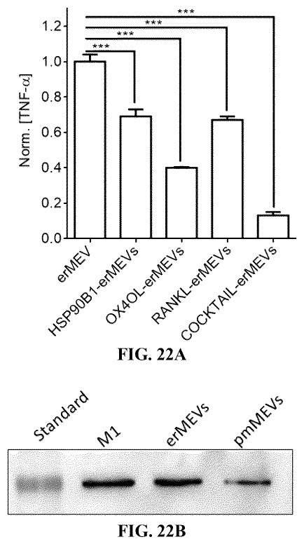

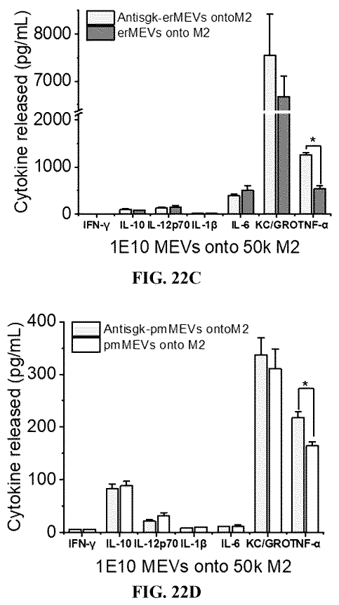

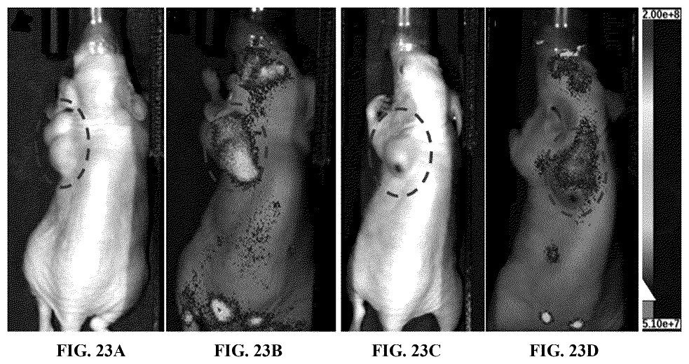

After identifying several ligands present on erMEVs and pmMEVs, their role on MEV-mediated macrophage repolarization was studied. erMEVs were separately incubated with primary neutralizing antibodies against HSP90B1, OX40L, RANKL, and a cocktail of antibodies against CCL5, HSP60, HSP90B1, OX40L, CD63, ICAM1, and TNF-α (FIG. 22A). The antibody treated vesicles were purified from the free antibodies by ultracentrifugation. Antibody treated erMEVs and unmodified erMEVs were incubated with 50,000 M2 BMDMs in culture. The immunomodulatory properties of antibody treated erMEVs were assessed by measuring the TNF-α secreted by M2 BMDMs that were incubated with MEVs.

HSP90B1 neutralizing antibody treated erMEVs (HSP90B1-erMEVs) elicited about 30% less proinflammatory response by M2 BMDMs (FIG. 22A). Similarly, OX40L neutralizing antibody treated erMEVs (OX40L-erMEVs), and RANKL neutralizing antibody treated erMEVs (RANKL-erMEVs), elicited about 40%, and 30% less proinflammatory response by M2 BMDMs (FIG. 22A). However, erMEVs treated with cocktail of antibodies against CCL5, HSP60, HSP90B1, OX40L, CD63, ICAM1, and TNF-α effectively reduced the pro-inflammatory response by M2 BMDMs to only 10% of what is observed for regular antibody non-treated erMEVs (FIG. 22A). These results demonstrate that upon blocking the activity of endogenous ligands present on erMEVs using respective neutralizing antibodies, these vesicles elicit significantly lower macrophage polarizing activity.

Based on these results it was hypothesized that the endogenous ligands that adorn the surface of erMEVs or pmMEVs interact with their corresponding membrane receptors present in M2 macrophages to elicit repolarization of M2 BMDMs towards M1 like macrophages. To test this hypothesis, differentially expressed proteins that are localized in the endoplasmic reticulum were identified using a series of bioinformatics analyses including mRNA analysis, identification of protein localization from annotations available in an online database, and network analysis. As described in Neupane, et al. (2023), an NCBI dataset (GEO accession number GSE57614) resulting from a transcriptomic profiling of human macrophages polarized to M1 and M2 states (Derlindati et al. (2015)); these were analyzed with the GEO2R statistical tool that includes the R/Bioconductor and Limma packages. Derlindati et al. (2015). Using the Benjamini & Hochberg procedure (False discovery rate) to adjust the p-value, a cutoff of 0.05 was selected for the adjusted p-value to generate a list of differentially-expressed genes (DEGs). The UniProt database was used to extract a list of proteins that are localized in the ER and cross-referenced this with the list of DEGs. This resulted in identifying 25 differentially expressed proteins located in ER membrane. It was determined that there were 16 up-regulated genes in M1 macrophages that also reside in ER membrane. Bateman, et al. (2021); Derlindati, et al. (2015). These include RSAD2, PTGS2, ELOVL7, TAP1, SLC8A3, IL15RA, CYP7B1, GRAMD1B, TAP2, PML, PANX1, APOL2, VRK2, INSIG2, HLA-C and ANTXR2. Similarly, 9 down-regulated genes were identified: B3GLCT, SSR1, PTGS1, OSBPL3, SLC37A2, SGK1, GRAMD4, FLRT2 and PPM1L.

Following the identification of the 25 DEGs, a computational approach was used (Neupane et al. (2023)), to identify the proteins, whose expression could elicit macrophage phenotype reprogramming. From the network analysis, it was found that SGK1 inhibits M1 and promotes M2. This is consistent with the mRNA data for SGK1 in M2 vs M1 analysis showed a fold change of 3.26 with an adjusted p-value of 0.04217. Likewise, in M0 vs M1 RNA analysis, the fold change is 5.1 with an adjusted p-value of 0.00866. These changes suggest that SGK1 is upregulated in resting (M0) and M2 macrophages relative to M1, thus raising the hypothesis that inhibiting SGK1 could promote M1.

Recent studies have also shown that SGK1 promotes M2 polarization and curbs inflammation through FOXO1 and STAT3. Ren, et al. (2021). Upon performing western blotting analysis for SGK1, both erMEVs and pmMEVs were found to express SGK1 protein (FIG. 22C). In addition, erMEVs were found to express higher levels of SGK1 compared to pmMEVs (FIG. 22C). To understand the impact of the presence of SGK1 on M2 to M1 polarization, anti SGK1 neutralizing antibody treated erMEVs and pmMEVs were generated and then incubated with antibody treated erMEVs or pmMEVs and unmodified erMEVs with 50,000 M2 BMDMs in culture. Interestingly, antiSGK1 antibody treated erMEVs or pmMEVs elicited greater TNF-α production compared to unmodified erMEVs or pmMEVs respectively. However, there was no significant difference in the production of other pro-inflammatory cytokines produced by MEVs treated M2 BMDMs. These results demonstrate that by altering the proteins present in MEVs the reprogramming capability of macrophage engineered vesicles can be changed. In addition, these results support the hypothesis that erMEVs or pmMEVs elicit repolarization of M2 BMDMs towards M1 like macrophages through ligand-receptor interactions.

### Example 31: In-Vivo Delivery of erMEVs and pmMEVs

A set of experiments was performed to determine if subcellular organelle specific nanovesicles exhibited targeting specificity to tumor tissue when delivered in a live animal. Delivering macrophage engineered vesicles would prove to be a better alternative over targeting specificity exhibiting cancer cell derived vesicles because of their inherent safety issues. erMEVs and pmMEVs generated from mouse bone marrow derived pro-inflammatory macrophages were isolated and labeled separately with a lipophilic dye, DiR. The free dye present in the solution was separated from the vesicles using size-exclusion PD MidiTrap columns. DiR is embedded in the membrane of the vesicles enabling them to be tracked to determine the distribution or localization of vesicles within the animal. A subcutaneous A549 tumor xenograft implanted mouse was housed until the tumor size reached at least 100 mm³. An equal number of DiR labeled erMEVs or pmMEVs (˜2×10¹⁰) were then injected through the tail veins of separate mice. After 72 hours, in vivo imaging was conducted using an IVIS whole animal imager. Both erMEVs and pmMEVs had localization to their respective tumor.

### Example 32: Organelle Specific MEVs for Macrophage Repolarization

Both endoplasmic reticulum and plasma membrane-specific macrophage-engineered vesicles (MEVs) can be generated with high yields using nitrogen cavitation. These vesicles mimic the properties of endogenously released exosomes in terms of size, stability, and the ability to reprogram macrophages. This work further demonstrates that, compared to plasma membrane specific MEVs, endoplasmic reticulum specific MEVs are more potent at reprogramming M2 macrophages towards M1-like phenotypes. It was found that erMEVs elicit better reprogramming of already polarized macrophages. Therapies could use MEVs as standalone therapy or in conjunction with existing chemo and immune therapeutics.

In addition, erMEVs have a greater ability to suppress the viability of cancer cells compared to pmMEVs. Organelle specific MEVs exhibit differences in their membrane proteins that may contribute to their ability to reprogram macrophages. Altering the activity of these proteins changes the ability of MEVs to reprogram macrophages. Under in vivo conditions, both erMEVs and pmMEVs exhibit similar tumor targeting properties. Overall, organelle specific MEVs offer an alternative to endogenously produced vesicles as a therapeutic tool for macrophage repolarization.

### Example 33: Animals

For organelle specific macrophage engineered vesicle studies, C57BL/6 wild-type mice aged between 2-5 months were used to extract bone marrow cells. Mice were taken proper care of by accommodating them in IVC cages and providing sufficient food and water. All the experiments were performed following the guidelines from NIH and were approved by the Institutional Animal Care and Use Committee at the University of Kentucky.

### Example 34: Primary Cell Culture

Bone marrow-derived macrophages (BMDMs) were obtained from 2-3 month old C57BL/6 wild type mice following Neupane, et al. (2021). Briefly, bone marrow derived monocytes isolated from femur and tibia of a mice were cultured in the RPMI1640 medium supplemented with 10% FBS, 5% penicillin/streptomycin (PS), 1% glutamine, 1% HEPES, 0.001% β-mercaptoethanol and 20% supernatant from sL929 cells until for 7 days to allow complete differentiation of monocytes into macrophages. Cells were re-plated and were stimulated to M1 (LPS (20 ng/mL)+IFN-γ (20 ng/mL)) or M2 (IL-4 (20 ng/mL)) macrophages.

### Example 35: Cell Lines

Human lung cancer (A549) cells were maintained in DMEM medium supplemented with 10% FBS and 1% PS. Similarly, the ovarian adenocarcinoma cells (Caov-3) from ATCC were maintained in DMEM medium supplemented with 10% FBS. Cells were maintained at 37° C. with 5% CO₂.

### Example 36: Vesicles Isolation

Endoplasmic reticulum (er) and plasma membrane (pm) specific macrophage engineered vesicles were generated according to Moonschi, et al. (2018). Briefly, fully differentiated pro-inflammatory (M1) macrophages were used to generate er-MEVs and pm-MEVs. Cells were first washed with PBS and resuspended in a hypotonic protease inhibitor solution (10 mM Tris-HCl, 10 mM NaCl, 1.5 mM MgCl₂, 0.2 mM CaCl₂, pH adjusted to 7.4, one protease inhibitor tablet per 10 ml buffer) and then swirled gently for 10 minutes under ice-cold condition to facilitate the swelling of the macrophages. The cell slurry was then transferred into a pre-chilled nitrogen cavitation vessel (Parr Instruments Company, IL, USA) on ice and subjected to a nitrogen gas pressure of 600 psi for 20 minutes. The pressure was rapidly released resulting in the fragmentation of the cells and the subsequent generation of vesicles. In an Ultra-Clear ultracentrifuge tube, 3 mL of the cell lysate was added then on top of the OptiPrep gradient consisting of 3 mL of each of the 30% (bottom), 20% (middle) and 10% (top) OptiPrep solutions. Ultracentrifuge tube containing OptiPrep gradient was fitted in a swing bucket rotor and centrifuged at 25,000 rpm (112,000×g) for 90 min at 4° C. Peristaltic pump was used to collect ER and PM fractions from the three layers of vesicles that were observed between cell lysate and 10% OptiPrep, 10% and 20% OptiPrep, and 20% and 30% OptiPrep after the centrifugation. ER and the plasma membrane fractions were diluted with 1×PBS buffer to 1:3 and centrifuged at 30,000 rpm (100,000×g) for 1 h at 4° C. with the fixed angle rotor. The pellets obtained after ultracentrifugation were washed with PBS before resuspending them in PBS.

### Example 37: Nanoparticle Tracking Analysis

M1 macrophages were nitrogen cavitated to generate erMEVs and pmMEVs as discussed in the previous section. The size distribution and the concentration of the vesicles samples were obtained from nanoparticle tracking analysis (NTA) using NanoSight NS300 equipped with NTA analytical software and a 488 nm laser. Briefly, MEVs samples were diluted 1000 times in ultrapure water and five 60-s videos were recorded for analysis. The NS-300 uses a blue laser (465 nm) that interacts with the vesicles, and the scattered light coming from the vesicles is then collected by the camera and analyzed by the NTA software. The image processing software tracks each erMEV or pmMEV moving under Brownian motion and relates this movement to a vesicle size using the Stokes-Einstein equation. Boriachek, et al. (2018).

### Example 38: In Vitro Delivery

erMEVs and pmMEVs were labeled with DiI as discussed in Neupane, et al. (2021). 1×10¹⁰ DiI labeled erMEVs and pmMEVs were separately added to 50,000 M2 BMDMs in culture and left them to incubate for various time points ranging from 0.5 hours to 2.5 hours. Next, the labeled vesicles from the cells were rinsed and the cells were imaged using a wide-field microscope. Images were analyzed with Image J software.

### Example 39: Western Blotting Analysis

Pellets of M1 cells, erMEVs, and pmMEVs were first lysed separately in the chilled RIPA buffer. The solutions were left to incubate separately in ice for 30 minutes and gently sonicated for 30 seconds prior to centrifuging them at 14000×g for 15 minutes. Pellets thus obtained were discarded keeping the supernatant for protein concentration analysis. 50 μg of the respective protein samples were first resolved on 12% lab-made polyacrylamide gels, then transferred onto a nitrocellulose membrane. The membrane was then blocked for 1 hour using 5% milk solution and then incubated for 1 hour with corresponding primary antibodies at room temperature with constant shaking. After washing and removal of the primary antibody, HRP-conjugated secondary antibody was added and incubated for 1 h at room temperature with constant shaking. Then, the membranes were washed again, and bands were visualized by chemiluminescent detection (Clarity, Bio-Rad) using Chemi-Doc system (Bio-Rad).

### Example 40: Cytokine Quantification

M1 bone marrow-derived macrophages (BMDMs) were used to generate erMEVs and pmMEVs. For pro-inflammatory cytokine quantification, the cell culture supernatant from stimulated cells, the cells treated with erMEVs, and the cells treated with pmMEVs was collected after 24 hours. Thus collected supernatant, also known as macrophage conditioned media (MCM) was stored at −80° C. until the analysis was done. A mouse pro-inflammatory seven-plex assay from Meso Scale Discovery (MSD) was performed following manufacturers protocol and as discussed in Neupane, et al. (2023).

### Example 41: Determination of Cell Viability

Alamar blue cell viability assay was performed to determine the percentage of live cells present in any cell culture condition. Briefly, the cell media present in each well of a 96 well plate was carefully replaced with 100 μL of optimem (Invitrogen) and 20 μL of alamar blue was added into each well of a 96-well plate. Cells were then left to incubate for 30-45 minutes. Tecan 96-well plate reader equipped with an excitation filer set to 535 nm and the emission filter set to 595 nm was used to measure the fluorescence developed in each well of a well plate.

### Example 42: Co-Culture of M2 BMDMs and Carcinoma Cells With MEVs

20,000 A549 cells were left to incubate with 40,000 M2 Macrophages for 12 hours at 37° C. After 12 hours, the old growth media was aspirated-off without disturbing the cells in culture and carefully exchanged with new media containing erMEVs or pmMEVs. Similar experiments were performed with ovarian adenocarcinoma cells (Caov3). Briefly, 20,000 Caov3 cells were co-cultured with 40,000 M2 BMDMs for 12 hours prior to exchanging media containing erMEVs and pmMEVs. After 48 hours of incubation at 37° C., the media was aspirated off and 100 μL of optimem was added followed by 20 μL of alamar blue for the cell viability assay. The TNF-α cytokine released co-culture experiments was analyzed using a V-plex proinflammatory panel 1 (mouse) kit from MSD. Briefly, macrophages were cultured in 96 well plates at a density of 40,000 and then stimulated towards M2 phenotype for 24 hours using IL-4 (20 ng/mL). A549 cells were then plated at a density of 20000 cells/well with M2 macrophages for 12 hours at 37° C. After 12 hours of incubation, the cell media was replaced for replating media containing an equal number of erMEVs and pmMEVs and the cells were left to incubate for 24 hours at 37° C. MCM was collected and TNF-α assay was performed following manufacturers protocol.

### Example 43: In Vivo Delivery

100 million M1 stimulated BMDMs were used to generate erMEVs and pmMEVs in the same manner as described in the previous section. The vesicle pellets were then resuspended in 2 mL of sucrose buffer (10 mM HEPES, 250 mM Sucrose, pH adjusted to 7.4). 5 μL of 2 mM DiR, a lipophilic dye, was added separately onto vesicle resuspensions and allowed to incubate for 30 minutes at 37° C. In an Ultra-Clear ultracentrifuge tube, 2 mL of the labeled vesicle resuspension was added then on top of the OptiPrep gradient consisting of 2 mL of each of the 50% (bottom), and 10% (top) OptiPrep solutions. Ultracentrifuge tube containing OptiPrep gradient was fitted in a swing bucket rotor and centrifuged at 25,000 rpm (112,000×g) for 60 min at 4° C. Peristaltic pump was then used to collect DiR labeled vesicles present between the gradients. Vesicles were further purified to remove any free dye present in the solution using size exclusion PD Miditrap colums (Cytiva) and following the manufacturers protocol. 100 μL of DiR labeled endoplasmic reticulum and plasma membrane specific vesicles were injected separately into the athymic nude mice via lateral tail veins. These mice had palpable tumor xenografts of volumes greater than 100 mm³. Mice were imaged 72 hours post injection using a LagoX small optical imager (Spectral Instruments) fitted with 710 nm excitation and 770 nm emission filters. Images were processed using Aura Imaging software (Spectral Instruments).

### Example 44: AFM Vesicle Imaging

M1 BMDMs were used to generate erMEVs and pmMEVs as described in the earlier sections. Both erMEVs and pmMEVs were first labeled with DiI to confirm their attachment on the glass bottom dishes. To fix the labeled vesicles on the glass bottom dishes, a series of steps were performed. At first, the glass bottom dishes were incubated with 2% APTES solution for 30 minutes at room temperature. The excessive APTES was removed by rinsing multiple times with ethanol. The glass bottom dish was treated with 100 μM BAM in PBS for 10 minutes. The glass bottom dish was then washed with milli-Q water and added 1×10¹⁰ erMEVs and pmMEVs into separate glass bottom dishes. The vesicles were allowed to immobilize on the glass bottom dishes and finally rinsed with PBS to remove any unbound vesicles. Vesicles attached to the glass bottom dishes were then imaged using an AFM microscope.

### Example 45: Pathway Analysis

It was hypothesized that the erMEVs would contain significantly different levels of proteins localized to the ER, which could potentiate M1 or diminish M2 responses. This entailed first analyzing mRNA (Derlindati, et al. (2015)) to identify differentially expressed genes (DEGs) in M1 as described in Neupane, et al. (2023). This produced 429 genes that are differentially expressed in M1 compared to both M0 and M2 macrophages. ER membrane localized proteins were searched for in the UniProt database and identified 10,324 annotations of ER proteins at the time. Of these, 2404 were unique and 1326 were localized to the membrane. The localized ER membrane proteins were then cross-referenced with the DEGs that were identified. As a result, 25 DEGs were found to be in the ER membrane, of which sixteen of these genes exhibited increased expression by fold change (FC) score which is calculated from log (M2/M1). Using the computational approach described in Neupane, et al. (2023), these 25 DEGs and ER-localized proteins were entered as input to identify as outputs pathways that promote M2 versus M1 polarization. The search uses a graph network to represent the highly connected signaling pathways that were previously procured for macrophages, for which nodes represent specific genes, while the edges denote direct or indirect interactions. The nodes and edges were weighted where mRNA data were available from the transcriptomic study. Derlindati, et al. (2015). The software for conducting these assays is provided free of charge at github.com/huskeypm/pkh-lab-analyses.

### Example 46: Statistical Analysis

Statistical analyses were carried out using Origin 2018. For all the experimental conditions, data were expressed as mean±standard error of the mean (SEM). At least three independent experiments were performed for each condition (n=3). A one-way ANOVA test was done to test for statistical significance. Results will be considered statistically significant at P-values less than or equal to 0.01.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Anderson, N. R.; Minutolo, N. G.; Gill, S.; Klichinsky, M. Cancer Res. 2021, 81, 1201.
2. Andreu, Z.; Yáñez-Mó, M. Front. Immunol. 2014, 5, 442.
3. Arora, S.; Dev, K.; Agarwal, B.; Das, P.; Syed, M. A. Immunobiology 2018, 223, 383.
4. Bateman, A.; Martin, M.-J.; Orchard, S.; et al. Nucleic Acids Res. 2021, 49, D480.
5. Bauer, S.; Kirschning, C. J.; Häcker, H.; et al. Proc. Natl. Acad. Sci. U.S.A. 2001, 98, 9237.
6. Besingi, R. N.; Clark, P. L. Nat. Protoc. 2015, 10, 2074.
7. Biswas, S. K.; Mantovani, A. Nat. Immunol. 2010, 11, 889.
8. Boriachek, K.; Islam, M. N.; Möller, A.; et al. Small 2018, 14, 1801709.
9. Boutilier, A. J.; Elsawa, S. F. Int. J. Mol. Sci. 2021, 22, 6995.
10. Butowski, N.; Colman, H.; De Groot, J. F.; et al. Neuro-Oncol. 2016, 18, 557.
11. Cheng, H.; Wang, Z.; Fu, L.; Xu, T. Front. Oncol. 2019, 9, 421.
12. Cheng, L.; Wang, Y.; Huang, L. Mol. Ther. 2017, 25, 1665.
13. Choo, Y. W.; Kang, M.; Kim, H. Y.; et al. ACS Nano 2018, 12, 8977.
14. Daniele, S. G.; Béraud, D.; Davenport, C.; et al. Sci. Signal. 2015, 8, ra45.
15. Deng, W.; Chen, W.; Clement, S.; et al. Nat. Commun. 2018, 9, 2713.
16. Derlindati, E.; Cas, A. D.; Montanini, B.; et al. PLOS One 2015, 10, e0119751.
17. Diaz, G.; Bridges, C.; Lucas, M.; et al. J. Vis. Exp. 2018, 134, 57467.
18. Dong, X.; Gao, J.; Zhang, C. Y.; et al. ACS Nano 2019, 13, 1272.
19. Dostert, C.; Grusdat, M.; Letellier, E.; Brenner, D. Physiol. Rev. 2019, 99, 115.
20. Dragoni, S.; Hudson, N.; Kenny, B.-A.; et al. J. Immunol. 2017, 198, 4074.
21. El-Say, K.; El-Sawy, H. Int. J. Pharm. 2017, 528, 675.
22. Fan, Z.; Wang, Y.; Li, L.; et al. ACS Nano 2022, 16, 16177.
23. Fantuzzi, L.; Belardelli, F.; Gessani, S. J. Leukoc. Biol. 2003, 74, 719.
24. Figenschau, S. L.; Knutsen, E.; Urbarova, I.; et al. Sci. Rep. 2018, 8, 11720.
25. Fishman, P.; Bar-Yehuda, S.; Liang, B. T.; Jacobson, K. A. Drug Discov. Today 2012, 17, 359.
26. Fiorentino, D. F.; Zlotnik, A.; Mosmann, T. R.; et al. J. Immunol. 1991, 147, 3815.
27. Gao, J.; Lee, M.; Dong, X.; Wang, Z. Methods Mol. Biol. 2022, 2394, 575.
28. Goh, W. J.; Zou, S.; Ong, W. Y.; et al. Sci. Rep. 2017, 7, 14322.
29. Gosset, P.; Tillie-Leblond, I.; Oudin, S.; et al. J. Allergy Clin. Immunol. 1999, 103, 289.
30. Guiducci, C.; Vicari, A. P.; Sangaletti, S.; et al. Cancer Res. 2005, 65, 3437.
31. Grohmann, U.; Belladonna, M. L.; Vacca, C.; et al. J. Immunol. 2001, 167, 221.
32. Gunassekaran, G. R.; Poongkavithai Vadevoo, S. M.; Baek, M. C.; Lee, B. Biomaterials 2021, 278, 121137.
33. Gschwandtner, M.; Derler, R.; Midwood, K. S. Front. Immunol. 2019, 10, 2759.
34. Hemmi, H.; Takeuchi, O.; Kawai, T.; et al. Nature 2000, 408, 740.
35. Harvey, B. T.; Fu, X.; Li, L.; et al. ACS Omega 2022, 7, 46222.
36. Hayden, M. S.; Ghosh, S. Semin. Immunol. 2014, 26, 253.
37. Hourani, T.; Holden, J. A.; Li, W.; et al. Front. Oncol. 2021, 11, 788365.
38. Hubbard, A. K.; Rothlein, R. Biol. Med. 2000, 28, 1379.
39. Jang, S. C.; Kim, O. Y.; Yoon, C. M.; et al. ACS Nano 2013, 11, 7698.
40. Jang, J.; Jeong, H.; Jang, E.; et al. Front. Plant Sci. 2022, 13, 1064412.
41. Kanehisa, M.; Furumichi, M.; Sato, Y.; et al. Nucleic Acids Res. 2023, 51, D587.
42. Kiepura, A.; Stachyra, K.; Wiśniewska, A.; et al. Int. J. Mol. Sci. 2021, 22, 9772.
43. Kim, H.; Wang, S. Y.; Kwak, G.; et al. Adv. Sci. 2019, 6, 1900513.
44. Kim, M. S.; Haney, M. J.; Zhao, Y.; et al. Nanomedicine 2016, 12, 655.
45. Kim, D. K.; Kang, B.; Kim, O. Y.; et al. J. Extracell. Vesicles 2013, 2, 20384.
46. Krishnamachari, Y.; Salem, A. K. Adv. Drug Deliv. Rev. 2009, 61, 205.
47. Lai, C. Y.; Yu, G. Y.; Luo, Y.; et al. Front. Immunol. 2019, 10, 179.
48. Li, M.; Sun, X.; Zhao, J.; et al. Cell Mol. Immunol. 2020, 17, 753.
49. Li, X.; Wang, S.; Zhu, R.; et al. J. Hematol. Oncol. 2016, 9, 42.
50. Li, Y.-J.; Wu, J.-Y.; Liu, J.; et al. J. Nanobiotechnol. 2021, 19, 242.
51. Lin, Y.; Xu, J.; Lan, H. J. Hematol. Oncol. 2019, 12, 76.
52. Liu, M.; O′Connor, R. S.; Trefely, S.; et al. Nat. Immunol. 2019, 20, 265.
53. Liu, P.; Chen, G.; Zhang, J. Molecules 2022, 27, 1372.
54. Liu, L.; Zheng, F. Onco Targets Ther. 2016, 9, 6083.
55. Liu, T.; Zhang, L.; Joo, D.; Sun, S.-C. Signal Transduct. Target Ther. 2017, 2, 17023.
56. Long, K. B.; Beatty, G. L. Oncoimmunology 2013, 2, e26860.
57. Lushpa, V. A.; Goncharuk, M. V.; Lin, C.; et al. Commun. Biol. 2021, 4, 1003.
58. Maguire, O.; Collins, C.; O'Loughlin, K.; et al. Cytometry A 2011, 79, 461.
59. Mantovani, A.; Sica, A.; Sozzani, S.; et al. Trends Immunol. 2004, 25, 677.
60. Miteva, L. D.; Stanilov, N. S.; Deliysky, T. S.; Stanilova, S. A. Tumour Biol. 2014, 35, 12655.
61. Mogensen, T. H. Clin. Microbiol. Rev. 2009, 22, 240.
62. Moonschi, F. H.; Effinger, A. K.; Zhang, X.; et al. Angew. Chem. Int. Ed. 2015, 54, 481.
63. Moonschi, F. H.; Fox-Loe, A. M.; Zhang, X.; Richards, C. I. Bio-protocol 2018, 8, e3054.
64 Mosser, D. M.; Edwards, J. P. Nat. Rev. Immunol. 2008, 8, 958.
65. Müller, E.; Christopoulos, P. F.; Halder, S.; et al. Front. Immunol. 2017, 8, 1383.
66. Neupane, K. R.; McCorkle, J. R.; Kopper, T. J.; et al. ACS Omega 2021, 6, 3847.
67. Neupane, K. R.; Ramon, G. S.; Harvey, B.; et al. Adv. Healthcare Mater. 2023, 12, e2301163.
68. Ozinsky, A.; Underhill, D. M.; Fontenot, J. D.; et al. Proc. Natl. Acad. Sci. U.S.A. 2000, 97, 13766.
69. Ratajczak, J.; Wysoczynski, M.; Hayek, F.; et al. Leukemia 2006, 20, 1487.
70. Rana, S.; Yue, S.; Stadel, D.; Zöller, M. Int. J. Biochem. Cell Biol. 2012, 44, 1574.
71. Ren, J.; Han, X.; Lohner, H.; et al. J. Immunol. 2021, 207, 268.
72. Ruytinx, P.; Proost, P.; Van Damme, J.; Struyf, S. Front. Immunol. 2018, 9, 1930.
73. Sands, H.; Gorey-Feret, L. J.; Cocuzza, A. J.; et al. Mol. Pharmacol. 1994, 45, 932.
74. Schweer, D.; McAtee, A.; Neupane, K.; et al. Cancers 2022, 14, 2220.
75. Sercombe, L.; Veerati, T.; Moheimani, F.; et al. Front. Pharmacol. 2015, 6, 286.
76. Sharma, S.; Rasool, H. I.; Palanisamy, V.; et al. ACS Nano 2010, 4, 1921.
77. Shen, J.; Sun, X.; Pan, B.; et al. Cancer Manag. Res. 2018, 10, 4217.
78. Sica, A.; Bronte, V. J. Clin. Invest. 2007, 117, 1155.
79. Smith, A. A. A.; Gale, E. C.; Roth, G. A.; et al. Biomacromolecules 2020, 21, 3704.
80. Snell, A. A.; Neupane, K. R.; McCorkle, J. R.; et al. ACS Omega 2019, 4, 12657.
81. Szklarczyk, D.; Gable, A. L.; Nastou, K. C.; et al. Nucleic Acids Res. 2021, 49, D605.
82. Talley, S.; Bonomo, R.; Gavini, C.; et al. Dis. Model Mech. 2022, 15, dmm049313.
83. Taylor, P.; Martinez-Pomares, L.; Stacey, M.; L. H-H. Annu. Rev. Immunol. 2005, 23, 901.
84. Termini, C. M.; Gillette, J. M. Front. Cell Dev. Biol. 2017, 5, 34.
85. Théry, C.; Ostrowski, M.; Segura, E. Nat. Rev. Immunol. 2009, 9, 581.
86. Tokunaga, R.; Zhang, W.; Naseem, M.; et al. Cancer Treat. Rev. 2018, 63, 40.
87. Trinchieri, G. Nat. Rev. Immunol. 2003, 3, 133.
88. UniProt Consortium. Nucleic Acids Res. 2021 Jan. 8;49 (D1): D480-D489.
89. U.S. Patent Application Publication No. 2023/0071507.
90. U.S. Patent Application Publication No. 2024/0228580.
91. Valadi, H.; Ekström, K.; Bossios, A.; et al. Nat. Cell Biol. 2007, 9, 654.
92. van Meer, G.; Voelker, D. R.; Feigenson, G. W. Nat. Rev. Mol. Cell Biol. 2008, 9, 112.
93. Wang, F.; Cerione, R. A.; Antonyak, M. A. STAR Protoc. 2021, 2, 100295.
94. Wang, Q.; Ren, J.; Morgan, S.; et al. PLOS One 2014, 9, e92053.
95. Wang, Y.; Tiruthani, K.; Li, S.; et al. Adv. Mater. 2021, 33, 2007603.
96. Wu, T.; Qi, Y.; Zhang, D.; et al. Adv. Funct. Mater. 2017, 27, 1703191.
97. Wu, Y.; Zhou, B. P. Br. J. Cancer 2010, 102, 639.
98. Xiao, M.; He, J.; Yin, L.; et al. Front. Immunol. 2021, 12, 799428.
99. Yang, M.; Liu, J.; Piao, C.; et al. Cell Death Dis. 2015, 6, e1780.
100. Yurkin, S. T.; Wang, Z. Nanomedicine 2017, 12, 2007.
101. Zarif, J. C.; Taichman, R. S.; Pienta, K. J. Oncoimmunology 2014, 3, e941734.
102. Zeng, Z.; Lan, T.; Wei, Y.; Wei, X. Genes Dis. 2022, 9, 12.
103. Zhang, B.; Kopper, T. J.; Liu, X.; et al. (Poster/abstract) 2019, 25.
104. Zhang, F.; Parayath, N. N.; Ene, C. I.; et al. Nat. Commun. 2019, 10, 3974.
105. Zhang, J.; Sun, X.; Li, H.; et al. J. Photoacoust. 2023, 30, 100462.
106. Zhang, L.; Wu, S.; Qin, Y.; et al. Nano Lett. 2019, 19, 4237.
107. Zhao, Y.-Y.; Zhu, Y.-C.; Chen, C.; et al. Gene 2022, 812, 146100.
108. Zhu, L.; Dong, D.; Yu, Z.-L.; et al. ACS Appl. Mater. Interfaces 2017, 9, 5100.
109. Zhu, Y.-Y.; Zhao, Y.-C.; Chen, C.; et al. Gene 2022, 812, 146100.
110. Zhuang, Y.; Zhao, X.; Yuan, B.; et al. J. Hepatocell. Carcinoma 2021, 8, 599.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of making programmed cell-derived vesicles (CDV), comprising:
(a) obtaining a donor cell from which the CDV will be generated, wherein
(b) the donor cell is isolated from an organelle of interest and/or the donor cell overexpresses a ligand of interest on the surface of the cell; and
(c) fragmenting the membrane of the donor cell and allowing the fragmented membrane to assemble into a CDV, wherein the CDV is an organelle-specific CDV and/or wherein the CDV displays the ligand of interest.

2. The method of claim 1, comprising:
(a) obtaining a donor cell from which the CDV will be generated;
(b) overexpressing a ligand of interest on the surface of the donor cell; and
(c) fragmenting the membrane of the donor cell and allowing the fragmented membrane to assemble into a CDV displaying the ligand of interest on its surface.

3. The method of claim 2, and further comprising overexpressing the ligand of interest by transfecting the donor cell with a plasmid for expressing the ligand of interest.

4. The method of claim 2, wherein the ligand of interest is a polarization-inducing ligand and/or a targeting-enhancing ligand.

5. The method of claim 2, wherein the ligand of interest is selected from the group consisting of CD54, TNF-α, CpG-ODN, ICOS, and combinations thereof.

6. The method of claim 2, wherein the ligand of interest is selected for interaction with a target of interest.

7. The method of claim 6, wherein the ligand of interest selectively binds the target of interest.

8. The method of claim 6, wherein the target of interest is in an in vivo environment.

9. The method of claim 1, comprising:
(a) isolating a donor cell from an organelle of interest, from which the CDV will be generated; and
(b) fragmenting the membrane of the donor cell and allowing the fragmented membrane to assemble into an organelle-specific CDV.

10. The method of claim 9, wherein the organelle of interest is selected from the group consisting of endoplasmic reticulum (ER), plasma membrane (PM), mitochondria.

11. The method of claim 9, wherein the organelle of interest is ER.

12. The method of claim 9, wherein the CDV is an ER-derived MEV (erMEV).

13. The method of claim 9, wherein a surface feature of the donor cell interacts with a target of interest.

14. The method of claim 13, wherein the target of interest is in an in vivo environment.

15. The method of claim 1, wherein the donor cell is a tumor cell, a dendritic cell, or a macrophage.

16. The method of claim 1, wherein the donor cell is a macrophage and further comprising polarizing the donor macrophage to a M1 phenotype.

17. The method of claim 1, and further comprising suspending the fragmented membrane in an assembly solution comprising cargo such that the CDV encapsulates the cargo during assembly.

18. A method of shifting a target macrophage phenotype to a M1 phenotype, comprising:
(a) contacting the target macrophage with a cell derived vesicle (CDV) displaying a ligand of interest, selected from the group consisting of CD54, TNF-α, CpG-ODN, ICOS, and combinations thereof;
(b) contacting the target macrophage with an of endoplasmic reticulum-derived macrophage-engineered vesicle (erMEV);
(c) contacting the target macrophage with an MEV derived from an M1 macrophage; or
(d) combinations thereof.

19. The method of claim 18, wherein the target macrophage is in an in vivo environment.

20. A cell-derived vesicle (CDV), comprising:
a membrane from a donor cell overexpressing a ligand of interest, such that the CDV displays the ligand of interest on its surface; or
a membrane from a donor cell isolated from an organelle of interest, which that the CDV is organelle-specific.
